SEQUENCE LISTING (87 sequences)

SEQ ID NO: 1 - protein, 343 aa, artificial
  extracellular SIRPA
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 2 - protein, 332 aa, artificial
  extracellular SIRPG
EEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATP

SEQ ID NO: 3 - protein, 794 aa, artificial
  anti-PD-1-CH1-KIH-FcG1m-(G4S)3-SIRPG (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATP

SEQ ID NO: 4 - protein, 794 aa, artificial
  Anti-PD1-FcG1m-(G4S)3-SIRPG
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATP

SEQ ID NO: 5 - protein, 805 aa, artificial
  Anti-PD1-FcG1m-(G4S)3-SIRPA
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 6 - protein, 805 aa, artificial
  Anti-PD1-KIH-FcG1m-(G4S)3-SIRPA (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 7 - protein, 566 aa, artificial
  anti-PD1-LC-SIRPG
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATP

SEQ ID NO: 8 - protein, 577 aa, artificial
  anti-PD1-LC-SIRPA
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 9 - protein, 447 aa, artificial
  anti-PD-1-CH1-KIH-FcG1m (Hole)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 447 aa, artificial
  Anti-PD-1-CH1-KIH-FcG1m (Knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, artificial
  Anti-PD-1-CL
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 220 aa, artificial
  Anti-PD-1-CH1
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 227 aa, artificial
  KIH-FcG1m (Hole)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 14 - protein, 227 aa, artificial
  KIH-FcG1m (Knob)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 794 aa, artificial
  anti-PD1-KIH-FcG1m-(G4S)3-SIRPG (knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATP

SEQ ID NO: 16 - protein, 9 aa, artificial
  LC CDR3
FQGTHVPNT

SEQ ID NO: 17 - protein, 117 aa, artificial
  HC variable domain
  MISC_FEATURE (105)..(105): X is X1, D or E
  MISC_FEATURE (106)..(106): X is X2, and is T, H, A, Y, N, E or S
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMXXWGQGTLVTVSS

SEQ ID NO: 18 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSS

SEQ ID NO: 19 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSS

SEQ ID NO: 20 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSS

SEQ ID NO: 21 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSS

SEQ ID NO: 22 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSS

SEQ ID NO: 23 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSS

SEQ ID NO: 24 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSS

SEQ ID NO: 25 - protein, 117 aa, artificial
  HC variable domain
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSS

SEQ ID NO: 26 - protein, 112 aa, artificial
  LC variable domain
  MISC_FEATURE (34)..(34): X is G or T
DVVMTQSPLSLPVTLGQPASISCRSSQSLVHANXNTYLEWYQQRPGQSPRLLIYKVSNRFSGVPDRFSGSGSGTDFTLKISRVEAEDVGVYYCFQGTHVPNTFGQGTKLEIK

SEQ ID NO: 27 - protein, 112 aa, artificial
  LC variable domain
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIK

SEQ ID NO: 28 - protein, 112 aa, artificial
  LC variable domain
DVVMTQSPLSLPVTLGQPASISCRSSQSLVHANTNTYLEWYQQRPGQSPRLLIYKVSNRFSGVPDRFSGSGSGTDFTLKISRVEAEDVGVYYCFQGTHVPNTFGQGTKLEIK

SEQ ID NO: 29 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 30 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDTWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 31 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMESWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 32 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 33 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 34 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 35 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 36 - protein, 444 aa, artificial
  HC
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDEWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 37 - protein, 219 aa, artificial
  LC
DVVMTQSPLSLPVTLGQPASISCRSSQSLVHANGNTYLEWYQQRPGQSPRLLIYKVSNRFSGVPDRFSGSGSGTDFTLKISRVEAEDVGVYYCFQGTHVPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 219 aa, artificial; identical to SEQ ID NO: 11
  LC

SEQ ID NO: 39 - protein, 327 aa, artificial
  HC constant domain
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 40 - protein, 107 aa, artificial [sequence omitted]

SEQ ID NO: 41 - protein, 30 aa, artificial
  HFR1
QIQLVQSGSELKKPGASVKVSCKASGYTFT

SEQ ID NO: 42 - protein, 14 aa, artificial
  HFR2
WVRQAPGQGLEWMG

SEQ ID NO: 43 - protein, 32 aa, artificial
  HFR3
RFVFSLDTSVSTAYLQISSLKAEDTAVYYCAR

SEQ ID NO: 44 - protein, 11 aa, artificial
  HFR4
WGQGTLVTVSS

SEQ ID NO: 45 - protein, 23 aa, artificial
  LFR1
DVVMTQSPLSLPVTLGQPASISC

SEQ ID NO: 46 - protein, 15 aa, artificial
  LFR2
WYQQRPGQSPRLLIY

SEQ ID NO: 47 - protein, 32 aa, artificial
  LFR3
GVPDRFSGSGSGTDFTLKISRVEAEDVGVYYC

SEQ ID NO: 48 - protein, 10 aa, artificial
  LFR4
FGQGTKLEIK

SEQ ID NO: 49 - protein, 19 aa, artificial
  signal peptide
MDWLWNLLFLMAAAQSIQA

SEQ ID NO: 50 - protein, 19 aa, artificial
  signal peptide
MKLPVRLLVLMFWIPASSS

SEQ ID NO: 51 - protein, 5 aa, artificial
  HC CDR1
HYAMN

SEQ ID NO: 52 - protein, 330 aa, artificial
  Heavy chain constant domain (IgG1m-N298A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 17 aa, artificial
  HC CDR2
WINTNTGEPTYAQGFTG

SEQ ID NO: 54 - protein, 8 aa, artificial
  HC CDR3
  MISC_FEATURE (7)..(7): X is X1 (D or E)
  MISC_FEATURE (8)..(8): X is X2 and is T, H, A, Y, N, E or S
EREPGMXX

SEQ ID NO: 55 - protein, 8 aa, artificial
  HC CDR3
EREPGMDS

SEQ ID NO: 56 - protein, 8 aa, artificial
  HC CDR3
EREPGMDT

SEQ ID NO: 57 - protein, 8 aa, artificial
  HC CDR3
EREPGMES

SEQ ID NO: 58 - protein, 8 aa, artificial
  HC CDR3
EREPGMDH

SEQ ID NO: 59 - protein, 8 aa, artificial
  HC CDR3
EREPGMDA

SEQ ID NO: 60 - protein, 8 aa, artificial
  HC CDR3
EREPGMDY

SEQ ID NO: 61 - protein, 8 aa, artificial
  HC CDR3
EREPGMDN

SEQ ID NO: 62 - protein, 8 aa, artificial
  HC CDR3
EREPGMDE

SEQ ID NO: 63 - protein, 16 aa, artificial
  LC CDR1
  MISC_FEATURE (11)..(11): X is G or T
RSSQSLVHANXNTYLE

SEQ ID NO: 64 - protein, 16 aa, artificial
  LC CDR1
RSSQSLVHANGNTYLE

SEQ ID NO: 65 - protein, 16 aa, artificial
  LC CDR1
RSSQSLVHANTNTYLE

SEQ ID NO: 66 - protein, 7 aa, artificial
  LC CDR2
KVSNRFS

SEQ ID NO: 67 - protein, 9 aa, artificial
  linker C
GGGGSGGGS

SEQ ID NO: 68 - protein, 5 aa, artificial
  linker G4S
GGGGS

SEQ ID NO: 69 - protein, 10 aa, artificial
  linker (G4S)2
GGGGSGGGGS

SEQ ID NO: 70 - protein, 15 aa, artificial
  linker (G4S)3
GGGGSGGGGSGGGGS

SEQ ID NO: 71 - protein, 444 aa, artificial
  HC anti-PD1 chimeric
QIQLVQSGPELKKPGETVKISCKASGYTFTHYGMNWVKQAPGKGLKWMGWINTNTGEPTYAEEFKGRFAFSLETSASAAFLQINNLKNEDTATYFCAKEREPGMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 72 - protein, 219 aa, artificial
  LC anti-PD1 chimeric
DVLLTQTPLSLPVSLGDQASISCRSSQNIVHANGNTYLEWYLQKPGQSPKLLIYKVSNRFSGVPDRFSGSGSGTDFTLKITRVEAEDLGVYYCFQGSHVPNTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - DNA, 2670 bp, artificial
  VH-VK
cagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatgacagatccagctggtgcagagcggctctgagctgaagaagccaggcgcttctgtgaaggtgtcctgcaaggccagcggctacaccttcacacactatgctatgaattgggtgagacaggctccaggacagggactggagtggatgggctggatcaacaccaatacaggcgagcctacctacgctcagggctttacaggccgcttcgtgttttctctggatacctccgtgagcacagcctatctgcagatctccagcctgaaggctgaggacaccgccgtgtactattgtgctagggagagggagccaggaatggataactggggacagggcaccctggtgacagtgtcttccgctagcaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaatga

SEQ ID NO: 74 - DNA, 660 bp, artificial
  VL-VD
gacgtggtcatgacacagagcccactgtctctgcctgtgaccctgggacagccagcctctatctcctgcagatccagccagtctctggtgcacgctaacaccaatacatacctggagtggtatcagcagaggccaggacagtccccaaggctgctgatctacaaggtgtccaacagattcagcggagtgccagaccgctttagcggatctggatccggaaccgacttcaccctgaagatctccagggtggaggctgaggatgtgggcgtgtactattgtttccagggcacccatgtgcctaatacatttggccagggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 75 - protein, 805 aa, artificial
  anti-PD1-KIH-FcG1m-(G4S)3-SIRPA (knob)
QIQLVQSGSELKKPGASVKVSCKASGYTFTHYAMNWVRQAPGQGLEWMGWINTNTGEPTYAQGFTGRFVFSLDTSVSTAYLQISSLKAEDTAVYYCAREREPGMDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIY

SEQ ID NO: 76 - protein, 120 aa, artificial
  VH anti-CD28
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS

SEQ ID NO: 77 - protein, 10 aa, artificial
  HCDR1 anti-CD28
GYTFTSYYIH

SEQ ID NO: 78 - protein, 17 aa, artificial
  HCDR2 anti-CD28
CIYPGNVNTNYNEKFKD

SEQ ID NO: 79 - protein, 11 aa, artificial
  HCDR3 anti-CD28
SHYGLDWNFDV

SEQ ID NO: 80 - protein, 107 aa, artificial
  VL anti-CD28
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 81 - protein, 11 aa, artificial
  LCDR1 anti-CD28
HASQNIYVWLN

SEQ ID NO: 82 - protein, 7 aa, artificial
  LCDR2 anti-CD28
KASNLHT

SEQ ID NO: 83 - protein, 9 aa, artificial
  LCDR3 anti CD-28
QQGQTYPYT

SEQ ID NO: 84 - protein, 228 aa, artificial
  Fc chain (Knob)
EDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 459 aa, artificial
  Heavy chain (Hole)
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSRTKLTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 86 - protein, 584 aa, artificial
  Light chain-SIRPa Bicki-CD28-SIRPa (monoTGN-SIRPa)
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKKLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKAAAGGGGSGGGGSGGGGSEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYAAAHHHHHH

SEQ ID NO: 87 - protein, 573 aa, artificial
  Bicki-CD28-SIRPg (monoTGN-SIRPg) Light chain-SIRPg
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKKLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKAAAGGGGSGGGGSGGGGSEEELQMIQPEKLLLVTVGKTATLHCTVTSLLPVGPVLWFRGVGPGRELIYNQKEGHFPRVTTVSDLTKRNNMDFSIRISSITPADVGTYYCVKFRKGSPENVEFKSGPGTEMALGAKPSAPVVLGPAARTTPEHTVSFTCESHGFSPRDITLKWFKNGNELSDFQTNVDPTGQSVAYSIRSTARVVLDPWDVRSQVICEVAHVTLQGDPLRGTANLSEAIRVPPTLEVTQQPMRVGNQVNVTCQVRKFYPQSLQLTWSENGNVCQRETASTLTENKDGTYNWTSWFLVNISDQRDDVVLTCQVKHDGQLAVSKRLALEVTVHQKDQSSDATPAAAHHHHHH

(19) United States
(12) Patent Application Publication
(10) Pub. No.: US 2024/0182572 A1
(43) Pub. Date: Jun. 6, 2024

(54) SCAFFOLD FOR BIFUNCTIONAL MOLECULES COMPRISING PD-1 OR CD28 AND SIRP BINDING DOMAINS

(71) Applicant: OSE IMMUNOTHERAPEUTICS, NANTES (FR)

(72) Inventors: NICOLAS POIRIER, NANTES (FR); AURORE MORELLO, NANTES (FR); CAROLINE MARY, NANTES (FR); JUSTINE DURAND, NANTES (FR)

(21) Appl. No.: 18/285,664

(22) PCT Filed: Apr. 8, 2022
(86) PCT No.: PCT/EP2022/059411

§ 371 Date: Oct. 5, 2023

(30) Foreign Application Priority Data
Apr. 9, 2021 (EP) 21305463.8

(51) Int. Cl. C07K 16/28; A61K 39/00; A61P 35/00; C07K 14/705

(52) U.S. Cl. CPC C07K 16/2818; A61P 35/00; C07K 14/70596; A61K 2039/505; C07K 2317/55; C07K 2317/565; C07K 2317/569; C07K 2317/622; C07K 2319/30

(57) ABSTRACT

The present invention relates to bifunctional molecules having a particular scaffold and their uses.

### FIELD OF THE INVENTION

The invention pertains to the field of immunotherapy. The present invention provides a new scaffold for bifunctional molecules and their uses in medicine.

### BACKGROUND OF THE INVENTION

Bifunctional molecules are currently the object of developments in immunology, especially in the field of oncology. Indeed, they bring novel pharmacological properties through the co-engagement of two targets, may increase the safety profile as compared to a combination of two distinct molecule thanks to a targeted relocation to the tumor and may potentially reduce development and manufacturing costs associated with single drug product. However, these molecules are advantageous but may also present several inconveniences. The design of bifunctional molecules need to imply several key attributes such as binding affinity and specificity, folding stability, solubility, pharmacokinetics, effector functions, compatibility with the attachment of additional domains and production yield and cost compatible with a clinical developments.

Bifunctional molecules based on an antibody antagonizing PD-1 and linked to SIRPα have been disclosed, in particular in WO 2020/127373.

However, there is still a strong need of improved scaffold for bifunctional molecules.

### SUMMARY OF THE INVENTION

The present invention relates to a bifunctional molecule having a particular scaffold and comprising a single monovalent antigen binding domain that binds a target specifically expressed on immune cells surface and a single immune-stimulating moiety. This scaffold is essentially made of a dimeric Fc domain, the single monovalent antigen binding domain that binds a target specifically expressed on immune cells surface linked at the N terminal end of one monomer of the Fc domain and either i) the single immune-stimulating moiety linked at the C terminal end of the same monomer of the Fc domain or ii) the single monovalent antigen binding domain comprises a heavy variable chain and a light variable chain and the single immune-stimulating moiety is linked at the C terminal end of the light chain of antigen binding domain.

This particular scaffold is associated with an improved pharmacokinetic profile. This improvement has been observed with bifunctional molecules comprising different a SIRPγ extracellular domain as immune-stimulating moiety. The improved pharmacokinetic profile is surprising because, in absence of the immune-stimulating moiety, the improvement is not observed for this scaffold. The bifunctional molecule with this particular scaffold are favorable to cis-targeting of the two targets on the same cells, allowing a selective delivery of the immune stimulating moiety to the targeted cells. Finally, surprisingly, the bifunctional molecules having a particular scaffold have a better productivity and avoid the side products due to chain mispairing.

Accordingly, the present invention relates to a bifunctional molecule comprising a single antigen binding domain that binds to a target specifically expressed on immune cells surface and a single immune-stimulating moiety,
the molecule comprises a first monomer comprising an antigen-binding domain covalently linked via C-terminal end to N-terminal end of a first Fc chain, optionally via a peptide linker, and a second monomer comprising a complementary second Fc chain devoid of antigen-binding domain and of the immune-stimulating moiety;
wherein either i) the immune-stimulating moiety is covalently linked to the C-terminal end of said first Fc chain, optionally via a peptide linker; or ii) the single antigen binding domain comprises a heavy variable chain and a light variable chain and the immune-stimulating moiety is covalently linked to the C-terminal end of the light chain;
wherein the target specifically expressed on immune cells surface is selected from the group consisting of

PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL2, and PDL1; and
wherein the immune-stimulating moiety is selected from the group consisting of SIRPγ, SIRPα, SIRPβ, CD80, CD86, LIGHT, CTLA-4, TIGIT, CD40L, OX40L, APRIL and GITRL or a fragment thereof comprising the extracellular part thereof or a variant thereof having at least 80% of identity with the wildtype protein or the extracellular part thereof.

In a particular aspect, the immune-stimulating moiety is linked at the C-terminal end of first Fc chain, preferably by its N-terminal end.

In a particular aspect, the immune-stimulating moiety is linked at the C-terminal end of the light chain, preferably by its N-terminal end.

In a particular aspect, the first Fc chain and the second Fc chain form a heterodimeric Fc domain, in particular a knob-into-hole heterodimeric Fc domain.

Optionally, the immune-stimulating moiety is selected from the group consisting of SIRPα, SIRPγ, and or a fragment thereof comprising the extracellular part thereof or a variant thereof having at least 80% of identity with the wildtype protein or the extracellular part thereof.

In an additional particular aspect, the immune-stimulating moiety is SIRPalpha or SIRPgamma, a fragment thereof, in particular comprising the extracellular domain thereof or a variant thereof having at least 80% of identity with SEQ ID NO: 1 or 2.

Optionally, the antigen-binding domain is a Fab domain, a Fab′, a single-chain variable fragment (scFV) or a single domain antibody (sdAb).

In a particular aspect, the target specifically expressed on immune cells surface is selected from the group consisting of PD-1, CD28, CTLA-4, BTLA, TIGIT, LAG3 and TIM3, preferably PD-1.

In a very particular aspect, the antigen binding domain is an anti-PD-1 antigen binding domain and comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or 65, a CDR2 of SEQ ID NO: 66 and a CDR3 of SEQ ID NO: 16.

In a very particular aspect, the antigen binding domain is an anti-CD28 antigen binding domain and comprises or consists essentially of: (i) a heavy chain comprising a CDR1 of SEQ ID NO: 77, a CDR2 of SEQ ID NO: 78 and a CDR3 of SEQ ID NO: 79; and (ii) a light chain comprising a CDR1 of SEQ ID NO: 81, a CDR2 of SEQ ID NO: 82 and a CDR3 of SEQ ID NO: 83.

The present invention also relates to an isolated nucleic acid sequence or a group of isolated nucleic acid molecules encoding the bifunctional molecule according to the present disclosure, and a host cell comprising the isolated nucleic acid(s).

The present invention further relates to a pharmaceutical composition comprising the bifunctional molecule, the nucleic acid(s) or the host cell according to the present disclosure, optionally with a pharmaceutically acceptable carrier.

Finally, the present invention relates to the bifunctional molecule, the nucleic acid(s), the host cell or the pharmaceutical composition according to the present disclosure for use as a medicament, especially for use in the treatment of a cancer or an infectious disease; the use of the bifunctional molecule, the nucleic acid(s), the host cell or the pharmaceutical composition according to the present disclosure for the manufacture of a medicament, especially for use in the treatment of a cancer or an infectious disease; and to a method of treating of a disease, especially a cancer or an infectious, in a subject comprising administering a therapeutically effective amount of the bifunctional molecule, the nucleic acid(s), the host cell or the pharmaceutical composition according to the present disclosure.

### BRIEF DESCRIPTION OF THE DRAWINGS

FIG. 1: Schematic representation of the different molecules used in the examples.

The anti PD-1*2/SIRP*1 was constructed with the SIRPg protein fused to the C terminal domain of a bivalent anti PD-1 antibody (Chain B). The chain A is a heavy chain of an anti-PD-1 antibody.

The anti-PD-1*1/SIRP*1 was constructed with SIRPg protein fused to the C terminal domain of a heavy chain B₂ of an anti PD-1 antibody (Chain B) via a linker (L). Chain B comprises the light chain B₁ and the heavy chain B₂. The chain A is a Fc domain that contains the CH1 CH2 and Hinge parts (H). Below the schematic representation of such antibodies is another representation of such construction, in which each chain and components of the molecule is further described.

All constructions were engineered with an lgG1 N298A isotype and amino acid sequences were mutated in the Fc portion to create a knob on the CH2 and CH3 of the Heavy chains A and a hole on the CH2 and CH3 of the Heavy chains B. All constructions also comprise a GGGGSGGGGSGGGGS linker (SEQ ID NO: 70) between the Fc domain and the SIRP fused protein.

The anti-CD28*1/SIRP*1 was constructed with a SIRP protein fused to the C terminal domain of a light chain B₁ of an anti PD-1 antibody (Chain B) via a linker (L). Chain B comprises the light chain B₁ and the heavy chain B₂. The chain A is a Fc domain that contains the CH1 CH2 and Hinge parts (H).

FIG. 2: Productivity of anti PD-1*1/SIRPg*1 bifunctional molecule in mammalian cells. CHO-S cells were transiently transfected with the DNA encoding for the anti PD-1*2/SIRPg*1 or the anti PD-1*1/SIRPg*1 molecules at a ratio (1:3:3; Chain A: Chain B: VL). Supernatant containing the antibodies were purified using Protein A chromatography. The quantity of bifunctional antibody obtained after purification was quantified by UV spectrometry (DO 280 nm) and normalized to the volume of production.

FIG. 3: Anti PD-1*1/SIRP*1 demonstrates efficient bind to human PD-1. PD-1 binding ELISA assay. Human recombinant PD-1 (rPD1) protein was immobilized, and anti PD-1*1/SIRPg*1 was added at different concentrations. Revelation was performed with an anti-human Fc antibody coupled to peroxidase. Colorimetry was determined at 450 nm using TMB substrate. All constructions tested comprise a GGGGSGGGGSGGGGS linker between the Fc and SIRP domain.

FIG. 4: Anti PD-1*1 SIRP*1 demonstrated better pharmacokinetics profile in vivo compared to the Anti PD-1*2 SIRP*1 and the Anti PD-1*2 SIRP*2 bifunctional molecule. C57BL/6 mice were intravenously injected with one dose (34 nmol/kg) of bifunctional anti PD-1/SIRP molecules. Antibodies concentration in the sera was quantified at multiple time points using an anti-human Fc specific ELISA. Left graph, data are represented in nanomolar concentration. Right graph, area under the curve was calculated for each construction to define the in vivo drug exposure.

FIG. 5: Pharmacokinetics study in mice after a single injection of anti PD-1 alone (Anti PD-1*1 and Anti PD-1*2). C57 BL6 Mice were injected with one dose (34 nmol/kg) of anti PD-1 antibody constructed with one anti PD-1 valency (anti PD-1*1 , dashed line) or 2 anti PD-1 valency (anti PD-1*2 ·, plain line) (FIG. 5A) Intravenous injection and (FIG. 5B) Intraperitoneal injection. Antibodies concentration in the sera was quantified at multiple time points using an anti-human Fc specific ELISA. Data are represented in nM concentration.

FIG. 6: Anti PD-1*1/SIRPg*1 molecules promote phagocytosis of CD47 + tumor cells by macrophages. Human M1 macrophages differentiated from peripheral blood monocytes (CD14+) were cultivated with CD47 negative or CD47 positive HUH7 hepatocarcinoma human cells (ratio 1:2) for 45 minutes with an isotype control, the anti PD-1*2 or the anti PD-1*1/SIRPg*1. Prior incubation, Macrophages and tumor cells were labelled with different cell tracker dyes (Cell tracker green or cell tracker red). Phagocytosis was quantified by flow cytometry and data were normalized to the isotype control group. Data represent mean +/− SD of 3-6 independent experiments. Statistical significance was calculated with a One Way Anova test and a Tukey's multiple comparisons test. **<0,0012, ***<0,0004.

FIG. 7: Anti CD28/SIRP bispecific antibodies efficiently bind to CD28. The binding of bispecific antibody was quantified using MSD technology. Human CD28-Fc was coated to the well (R&D #342-CD) at 5 μg/ml (MSD plate), then anti-CD28/SIRPα (▴) or anti CD28/SIRPg (▾) were added at serial dilutions. As positive control an anti-CD28 with 2 paratopes (Δ) was tested and an isotype was used as negative control (grey line). Binding of antibodies was revealed using an anti-human sulfo-tag Kappa antibody (dilution 1 to 1000).

FIG. 8: Anti CD28/SIRP bispecific antibodies have trans activation properties to promote T cell activation. Jurkat CD28+ T cell line (1^{e}5 cells) were plated on a CD3 (A) or CD3+ CD47 Fc (B) coated plate. CD3 (OKT3 1 μg/mL coating) and CD47 -Fc (Sinobiological #12283 10 μg/mL coating). Bispecific antibodies, SIRPA or SIRPG were incubated at 100 nM. AncCD28.1 (superagonist anti-CD28 antibody) was used as positive control of IL-2 secretion. 48 h after stimulation, IL-2 secretion was quantified by ELISA in culture supernatant.

### DETAILED DESCRIPTION OF THE INVENTION

### Introduction

The present invention relates to a bifunctional molecule having a particular scaffold and comprising a single monovalent antigen binding domain that binds a target specifically expressed on immune cells surface and a single immune-stimulating moiety. This scaffold is essentially made of a dimeric Fc domain, a single monovalent antigen binding domain that binds a target specifically expressed on immune cells surface linked at the N terminal end of one monomer of the Fc domain and a single immune-stimulating moiety linked at the C terminal end of the monomer of the Fc domain or the light chain when the antigen binding domain comprises a heavy variable chain and a light variable chain.

These novel bifunctional molecules present, among other advantages, an improved pharmacokinetic profile, and a better productivity.

### Definitions

In order that the present invention may be more readily understood, certain terms are defined hereafter. Additional definitions are set forth throughout the detailed description.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art.

As used herein, the term “antibody” describes a type of immunoglobulin molecule and is used in its broadest sense. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. The heavy-chain constant domains that correspond to the different classes of immunoglobulins are called alpha, delta, epsilon, gamma, and mu, respectively. Unless specifically noted otherwise, the term “antibody” includes intact immunoglobulins and “antibody fragment” or “antigen binding fragment” (such as Fab, Fab′, F(ab′)2, Fv), single chain (scFv), mutants thereof, molecules comprising an antibody portion, diabodies, linear antibodies, single chain antibodies, and any other modified configuration of the immunoglobulin molecule that comprises an antigen recognition site of the required specificity, including glycosylation variants of antibodies, amino acid sequence variants of antibodies. Preferably, the term antibody refers to a humanized antibody.

An “antibody heavy chain” as used herein, refers to the larger of the two types of polypeptide chains present in antibody conformations. The CDRs of the antibody heavy chain are typically referred to as “HCDR1”, “HCDR2” and “HCDR3”. The framework regions of the antibody heavy chain are typically referred to as “HFR1”, “HFR2”, “HFR3” and “HFR4”.

An “antibody light chain,” as used herein, refers to the smaller of the two types of polypeptide chains present in antibody conformations; K and A light chains refer to the two major antibody light chain isotypes. The CDRs of the antibody light chain are typically referred to as “LCDR1”, “LCDR2” and “LCDR3”. The framework regions of the antibody light chain are typically referred to as “LFR1”, “LFR2”, “LFR3” and “LFR4”.

As used herein, an “antigen-binding fragment” or “antigen-binding domain” of an antibody means a part of an antibody, i.e. a molecule corresponding to a portion of the structure of the antibody of the invention, that exhibits antigen-binding capacity for a particular antigen, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragment that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies. Antigen-binding fragments of antibodies are fragments which comprise their hypervariable domains designated CDRs (Complementary Determining Regions) or part(s) thereof.

As used herein, the term “humanized antibody” is intended to refer to antibodies in which CDR sequences derived from the germline of another mammalian species, such as a mouse, have been grafted onto human framework sequences (e.g. chimeric antibodies that contain minimal sequence derived from a non-human antibody). A “humanized form” of an antibody, e.g., a non-human antibody, also refers to an antibody that has undergone humanization. A humanized antibody is generally a human immunoglobulin (recipient antibody) in which residues from one or more CDRs are replaced by residues from at least one CDR of a non-human antibody (donor antibody) while maintaining the desired specificity, affinity, and capacity of the original antibody. Additional framework region modifications may be made within the human framework sequences. Preferably humanized antibody has a T20 humanness score greater than 80%, 85% or 90%. “Humanness” of an antibody can for example be measured using the T20 score analyzer to quantify the humanness of the variable region of antibodies as described in Gao S H, Huang K, Tu H, Adler A S. BMC Biotechnology. 2013: 13:55 or via a web-based tool to calculate the T20 score of antibody sequences using the T20 Cutoff Human Databases: http://abAnalyzer.lakepharma.com.

By “chimeric antibody” is meant an antibody made by combining genetic material from a nonhuman source, preferably such as a mouse, with genetic material from a human being. Such antibody derives from both human and non-human antibodies linked by a chimeric region. Chimeric antibodies generally comprise constant domains from human and variable domains from another mammalian species, reducing the risk of a reaction to foreign antibodies from a non-human animal when they are used in therapeutic treatments.

As used herein, the terms “fragment crystallizable region” “Fc region” or “Fc domain” are interchangeable and refers to the tail region of an antibody that interacts with cell surface receptors called Fc receptors. The Fc region or domain is typically composed of two domains, optionally identical, derived from the second and third constant domains of the antibody's two heavy chains (i.e. CH2 and CH3 domains). Portion of the Fc domain refers to the CH2 or the CH3 domain. Optionally, the Fc region or domain may optionally comprise all or a portion of the hinge region between CH1 and CH2. Accordingly, the Fc domain may comprise the hinge, the CH2 domain and the CH3 domain. Optionally, the Fc domain is that from IgG1, IgG2, IgG3 or IgG4, optionally with IgG1 hinge-CH2-CH3 and IgG4 hinge-CH2-CH3.

In the context of lgG antibodies, the lgG isotypes each have three CH regions. Accordingly, “CH” domains in the context of lgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat.

By “amino acid change” or “amino acid modification” is meant herein a change in the amino acid sequence of a polypeptide. “Amino acid modifications” include substitution, insertion and/or deletion in a polypeptide sequence. By “amino acid substitution” or “substitution” herein is meant the replacement of an amino acid at a particular position in a parent polypeptide sequence with another amino acid. By “amino acid insertion” or “insertion” is meant the addition of an amino acid at a particular position in a parent polypeptide sequence. By “amino acid deletion” or “deletion” is meant the removal of an amino acid at a particular position in a parent polypeptide sequence. The amino acid substitutions may be conservative. A conservative substitution is the replacement of a given amino acid residue by another residue having a side chain (“R-group”) with similar chemical properties (e.g., charge, bulk and/or hydrophobicity). As used herein, “amino acid position” or “amino acid position number” are used interchangeably and refer to the position of a particular amino acid in an amino acids sequence, generally specified with the one letter codes for the amino acids. The first amino acid in the amino acids sequence (i.e. starting from the N terminus) should be considered as having position 1.

A conservative substitution is the replacement of a given amino acid residue by another residue having a side chain (“R-group”) with similar chemical properties (e.g., charge, bulk and/or hydrophobicity). In general, a conservative amino acid substitution will not substantially change the functional properties of a protein. Conservative substitutions and the corresponding rules are well-described in the state of the art. For instance, conservative substitutions can be defined by substitutions within the groups of amino acids reflected in the following tables:

TABLE A
Amino Acid Residue

| Amino Acid groups | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

TABLE B
Alternative Conservative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

TABLE C
Further Alternative Physical and Functional
Classifications of Amino Acid Residues

| | |
|---|---|
| Alcohol group-containing residues | S and T |
| Aliphatic residues | I, L, V, and M |
| Cycloalkenyl-associated residues | F, H, W, and Y |
| Hydrophobic | A, C, F, G, H, I, L, |
| residues | M, R, T, V, W, and Y |
| Negatively charged residues | D and E |
| Polar residues | C, D, E, H, K, N, Q, R, S, and T |
| Small residues | A, C, D, G, N, P, S, T, and V |
| Very small residues | A, G, and S |
| Residues involved | A, C, D, E, G, H, K, |
| in turn formation | N, Q, R, S, P, and T |
| Flexible residues | E, Q, T, K, S, G, P, D, E, and R |

As used herein, the “sequence identity” between two sequences is described by the parameter “sequence identity”, “sequence similarity” or “sequence homology”. For purposes of the present invention, the “percentage identity” between two sequences (A) and (B) is determined by comparing the two sequences aligned in an optimal manner, through a window of comparison. Said alignment of sequences can be carried out by well-known methods in the art, for example, using the algorithm for global alignment of Needleman-Wunsch. Protein analysis software matches similar sequences using measures of similarity assigned to various substitutions, deletions and other modifications, including conservative amino acid substitutions. Once the total alignment is obtained, the percentage of identity can be obtained by dividing the full number of identical amino acid residues aligned by the full number of residues contained in the longest sequence between the sequence (A) and (B). Sequence identity is typically determined using sequence analysis software. For comparing two amino acid sequences, one can use, for example, the tool “Emboss needle” for pairwise sequence alignment of proteins providing by EMBL-EBI and available on:

www.ebi.ac.uk/Tools/services/web/toolform.ebi?tool=emboss_needle&context=protein, for example using default settings: (I) Matrix: BLOSUM62, (ii) Gap open: 10, (iii) gap extend: 0.5, (iv) output format: pair, (v) end gap penalty: false, (vi) end gap open: 10, (vii) end gap extend: 0.5.

Alternatively, Sequence identity can also be typically determined using sequence analysis software Clustal Omega using the HHalign algorithm and its default settings as its core alignment engine. The algorithm is described in Söding, J. (2005) ‘Protein homology detection by HMM-HMM comparison’. Bioinformatics 21, 951-960, with the default settings.

The terms “derive from” and “derived from” as used herein refers to a compound having a structure derived from the structure of a parent compound or protein and whose structure is sufficiently similar to those disclosed herein and based upon that similarity, would be expected by one skilled in the art to exhibit the same or similar properties, activities and utilities as the claimed compounds.

As used herein, a “pharmaceutical composition” refers to a preparation of one or more of the active agents, such as comprising a bifunctional molecule according to the invention, with optional other chemical components such as physiologically suitable carriers and excipients. The purpose of a pharmaceutical composition is to facilitate administration of the active agent to an organism. Compositions of the present invention can be in a form suitable for any conventional route of administration or use. In one aspect, a “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. An “acceptable vehicle” or “acceptable carrier” as referred to herein, is any known compound or combination of compounds that are known to those skilled in the art to be useful in formulating pharmaceutical compositions.

“An effective amount” or a “therapeutic effective amount” as used herein refers to the amount of active agent required to confer therapeutic effect on the subject, either alone or in combination with one or more other active agents, e.g. the amount of active agent that is needed to treat the targeted disease or disorder, or to produce the desired effect. The “effective amount” will vary depending on the agent(s), the disease and its severity, the characteristics of the subject to be treated including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

As used herein, the term “medicament” refers to any substance or composition with curative or preventive properties against disorders or diseases.

The term “treatment” refers to any act intended to ameliorate the health status of patients such as therapy, prevention, prophylaxis and retardation of the disease or of the symptoms of the disease. It designates both a curative treatment and/or a prophylactic treatment of a disease. A curative treatment is defined as a treatment resulting in cure or a treatment alleviating, improving and/or eliminating, reducing and/or stabilizing a disease or the symptoms of a disease or the suffering that it causes directly or indirectly. A prophylactic treatment comprises both a treatment resulting in the prevention of a disease and a treatment reducing and/or delaying the progression and/or the incidence of a disease or the risk of its occurrence. In certain aspects, such a term refers to the improvement or eradication of a disease, a disorder, an infection or symptoms associated with it. In other aspects, this term refers to minimizing the spread or the worsening of cancers. Treatments according to the present invention do not necessarily imply 100% or complete treatment. Rather, there are varying degrees of treatment of which one of ordinary skill in the art recognizes as having a potential benefit or therapeutic effect. Preferably, the term “treatment” refers to the application or administration of a composition including one or more active agents to a subject who has a disorder/disease.

As used herein, the terms “disorder” or “disease” refer to the incorrectly functioning organ, part, structure, or system of the body resulting from the effect of genetic or developmental errors, infection, poisons, nutritional deficiency or imbalance, toxicity, or unfavorable environmental factors. Preferably, these terms refer to a health disorder or disease e.g. an illness that disrupts normal physical or mental functions. More preferably, the term disorder refers to immune and/or inflammatory diseases that affect animals and/or humans, such as cancer.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT) and myeloid-derived cells (neutrophil, eosinophil, basophil, monocyte, macrophage, dendritic cells). In particular, the immune cell can be selected in the non-exhaustive list comprising B cells, T cells, in particular CD4+ T cells and CD8+ T cells, NK cells, NKT cells, APC cells, dendritic cells and monocytes. “T cell” as used herein includes for example CD4+ T cells, CD8+ T cells, T helper 1 type T cells, T helper 2 type T cells, T helper 17 type T cells and inhibitory T cells.

As used herein, the term “T effector cell”, “T eff” or “effector cell” describes a group of immune cells that includes several T cells types that actively respond to a stimulus, such as co-stimulation. It particularly includes T cells which function to eliminate antigen (e.g., by producing cytokines which modulate the activation of other cells or by cytotoxic activity). It notably includes CD4+, CD8+, cytotoxic T cells and helper T cells (Th1 and Th2).

The term “exhausted T cell” refers to a population of T cell in a state of dysfunction (i.e. “exhaustion”). T cell exhaustion is characterized by progressive loss of function, changes in transcriptional profiles and sustained expression of inhibitory receptors. Exhausted T cells lose their cytokines production capacity, their high proliferative capacity and their cytotoxic potential, which eventually leads to their deletion. Exhausted T cells typically indicate higher levels of CD43, CD69 and inhibitory receptors combined with lower expression of CD62L and CD127.

The term “immune response” refers to the action of, for example, lymphocytes, antigen presenting cells, phagocytic cells, granulocytes, and soluble macromolecules produced by the above cells or the liver (including antibodies, cytokines, and complements) that results in selective damage to, destruction of, or elimination from the human body of invading pathogens, cells or tissues infected with pathogens, cancerous cells, or, in cases of autoimmunity or pathological inflammation, normal human cells or tissues.

The term “antagonist” as used herein, refers to a substance that blocks or reduces the activity or functionality of another substance. Particularly, this term refers to an antibody that binds to a cellular receptor (e.g. PD-1) as a reference substance (e.g. PD-L1 and/or PD-L2), preventing it from producing all or part of its usual biological effects (e.g. the creation of an immune suppressive microenvironment). The antagonist activity of a humanized antibody according to the invention may be assessed by competitive ELISA.

The term “agonist” as used herein, refers to a substance that activates the functionality of an activating receptor. Particularly, this term refers to an antibody that binds to a cellular activating receptor as a reference substance, and have at least partially the same effect of the biologically natural ligand (e.g. inducing the activatory effect of the receptor).

Pharmacokinetics (PK) refers to the movement of drugs through the body, whereas pharmacodynamics (PD) refers to the body's biological response to drugs. PK describes a drug's exposure by characterizing absorption, distribution, bioavailability, metabolism, and excretion as a function of time. PD describes drug response in terms of biochemical or molecular interactions. PK and PD Analyses are used to characterize drug exposure, predict and assess changes in dosage, estimate rate of elimination and rate of absorption, assess relative bioavailability / bioequivalence of a formulation, characterize intra- and inter-subject variability, understand concentration-effect relationships, and establish safety margins and efficacy characteristics. By “improving PK” it is meant that one of the above characteristics is improved, for example, such as an increased half-life of the molecule, in particular a longer serum half-life of the molecule when injected to a subject.

As used herein, the terms “pharmacokinetics” and “PK” are used interchangeably and refer to the fate of compounds, substances or drugs administered to a living organism. Pharmacokinetics particularly comprise the ADME or LADME scheme, which stands for Liberation (i.e. the release of a substance from a composition), Absorption (i.e. the entrance of the substance in blood circulation), Distribution (i.e.

dispersion or dissemination of the substance through the body) Metabolism (i.e. transformation or degradation of the substance) and Excretion (i.e. the removal or clearance of the substance from the organism). The two phases of metabolism and excretion can also be grouped together under the title elimination. Different pharmacokinetics parameters can be monitored by the man skilled in the art, such as elimination half-life, elimination constant rate, clearance (i.e. the volume of plasma cleared of the drug per unit time), Cmax (Maximum serum concentration), and Drug exposure (determined by Area under the curve, see Scheff et al, Pharm Res. 2011 May;28(5):1081-9) among others.

As used herein, the term “isolated” indicates that the recited material (e.g., antibody, polypeptide, nucleic acid, etc.) is substantially separated from, or enriched relative to, other materials with which it occurs in nature. Particularly, an “isolated” antibody is one which has been identified and separated and/or recovered from a component of its natural environment.

The term “and/or” as used herein is to be taken as specific disclosure of each of the two specified features or components with or without the other. For example, “A and/or B” is to be taken as specific disclosure of each of (i) A, (ii) B and (iii) A and B, just as if each is set out individually.

The term “a” or “an” can refer to one of or a plurality of the elements it modifies (e.g., “a reagent” can mean one or more reagents) unless it is contextually clear either one of the elements or more than one of the elements is described.

The term “about” as used herein in connection with any and all values (including lower and upper ends of numerical ranges) means any value having an acceptable range of deviation of up to +/−10% (e.g., +/−0.5%, +/−1%, +/−1 .5%, +/−2%, +/−2.5%, +/−3%, +/−3.5%, +/−4%, +/−4.5%, +/−5%, +/−5.5%, +/−6%, +/−6.5%, +/−7%, +/−7.5%, +/−8%, +/−8.5%, +/−9%, +/−9.5%). The use of the term “about” at the beginning of a string of values modifies each of the values (i.e. “about 1, 2 and 3” refers to about 1, about 2 and about 3). Further, when a listing of values is described herein (e.g. about 50%, 60%, 70%, 80%, 85% or 86%) the listing includes all intermediate and fractional values thereof (e.g., 54%, 85.4%).

### Bifunctional Molecules

The present invention relates to a bifunctional molecule having a scaffold with improved properties.

More particularly, the present invention relates to a bifunctional molecule having a particular scaffold and comprising a single monovalent antigen binding domain that binds a target specifically expressed on immune cells surface and a single immune-stimulating moiety. This scaffold is essentially made of a dimeric Fc domain, a single monovalent antigen binding domain that binds a target specifically expressed on immune cells surface linked at the N terminal end of one monomer of the Fc domain and either i) a single immune-stimulating moiety linked at the C terminal end of the same monomer of the Fc domain, or ii) the single monovalent antigen binding domain comprises a heavy variable chain and a light variable chain and the single immune-stimulating moiety is linked at the C terminal end of the light chain of said antigen binding domain, and optionally peptide linkers.

In a particular aspect, the bifunctional molecule comprises a first monomer comprising an antigen-binding domain covalently linked to a first Fc chain optionally via a peptide linker, said first Fc chain being covalently linked to the immune-stimulating moiety, optionally via a peptide linker, and a second monomer comprising a complementary second Fc chain, devoid of antigen-binding domain and of immune-stimulating moiety, said first and second Fc chains forming a dimeric Fc domain.

In an alternative aspect, the bifunctional molecule comprises a first monomer comprising an antigen-binding domain covalently linked to a first Fc chain optionally via a peptide linker, a second monomer comprising a complementary second Fc chain, devoid of antigen-binding domain and of immune-stimulating moiety, said first and second Fc chains forming a dimeric Fc domain, and the single monovalent antigen binding domain comprises a heavy variable chain and a light variable chain and the single immune-stimulating moiety is linked at the C terminal end of the light chain of said antigen binding domain.

Accordingly, two monomers comprise each one a Fc chain, the Fc chains being able to form a dimeric Fc domain. In one aspect, the dimeric Fc fusion protein is a homodimeric Fc domain. In another aspect, the dimeric Fc fusion protein is a heterodimeric Fc domain.

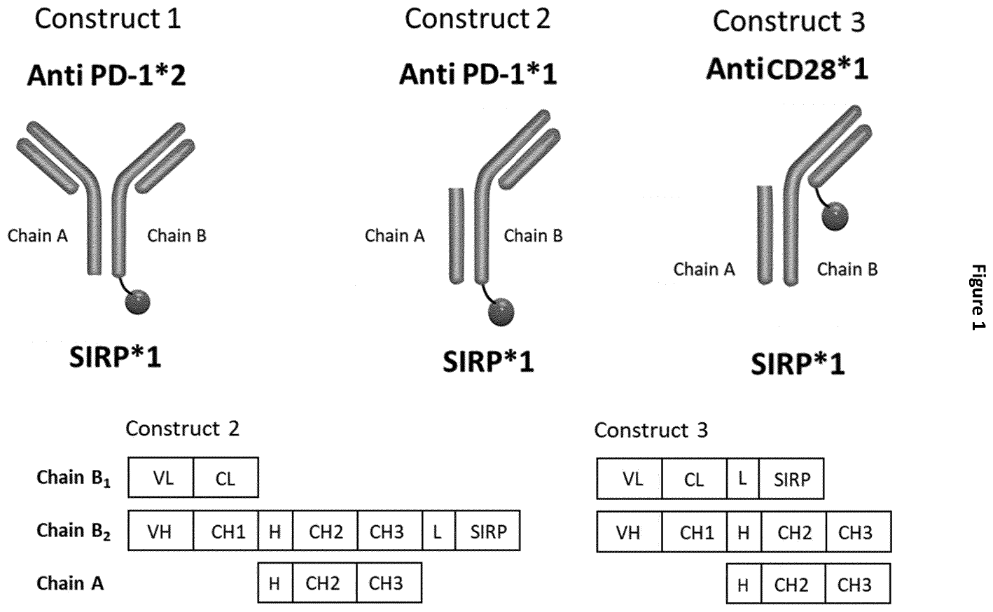

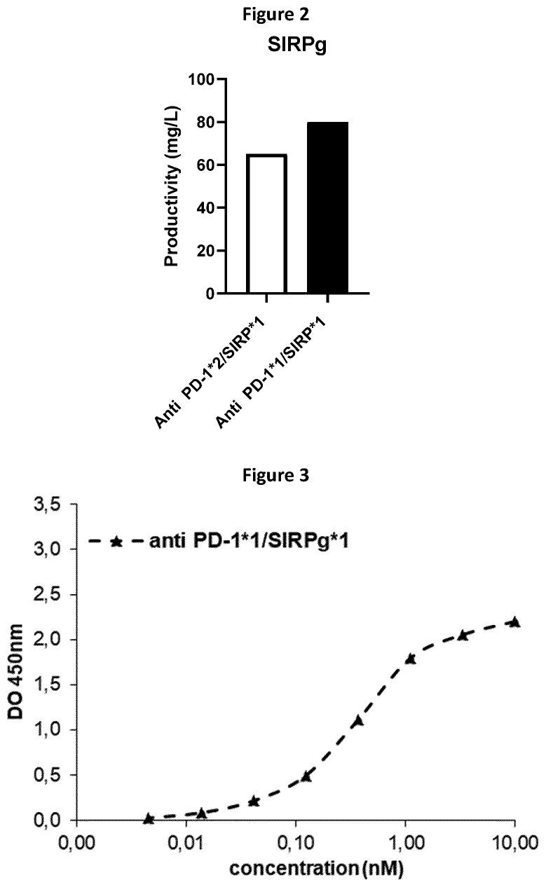

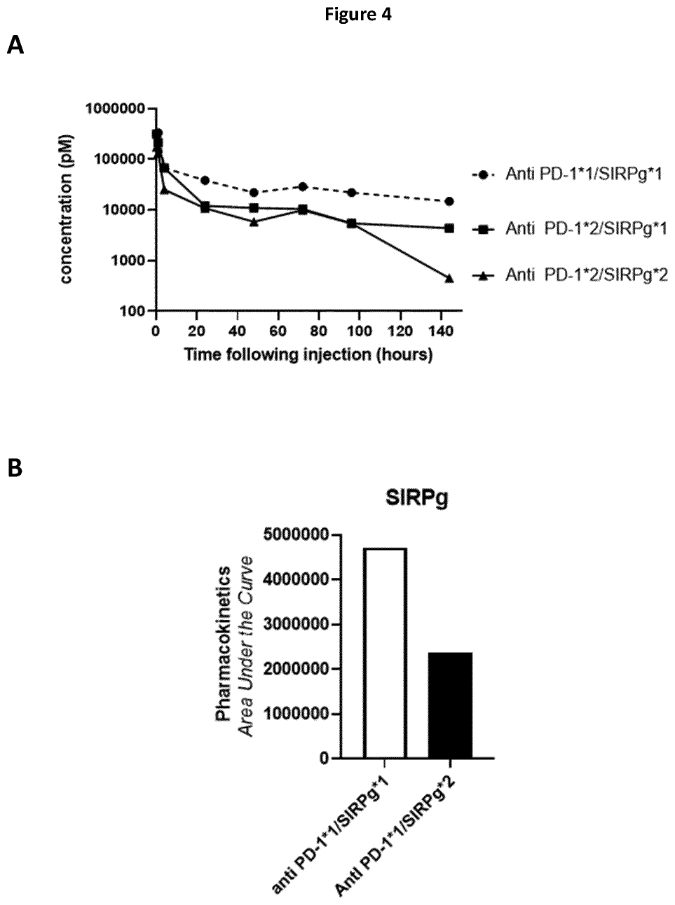

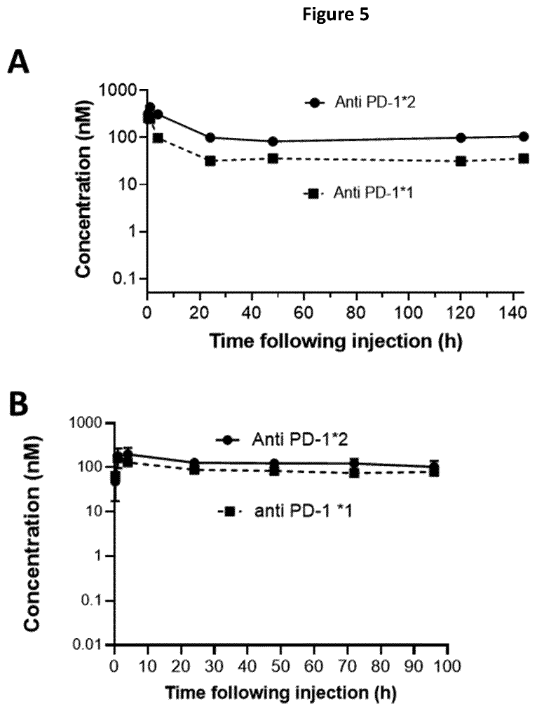

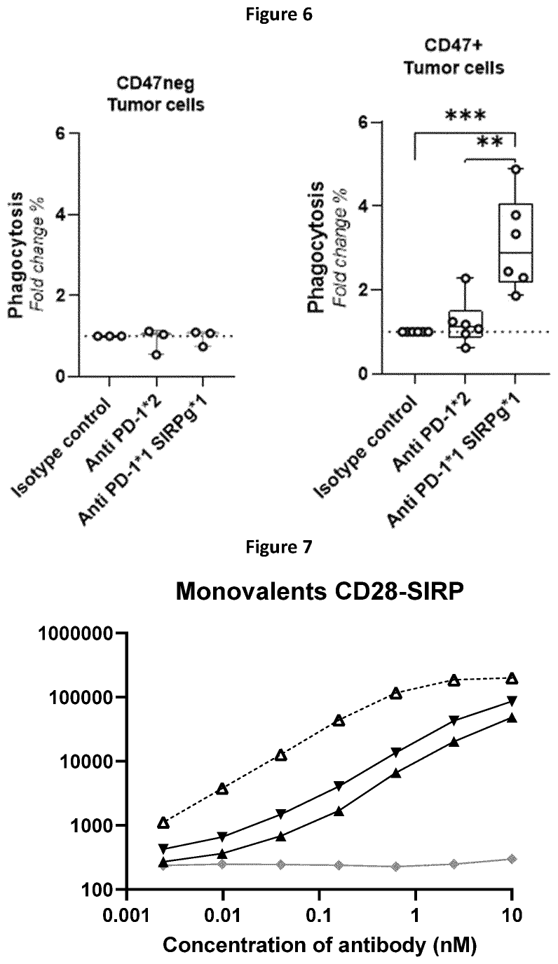

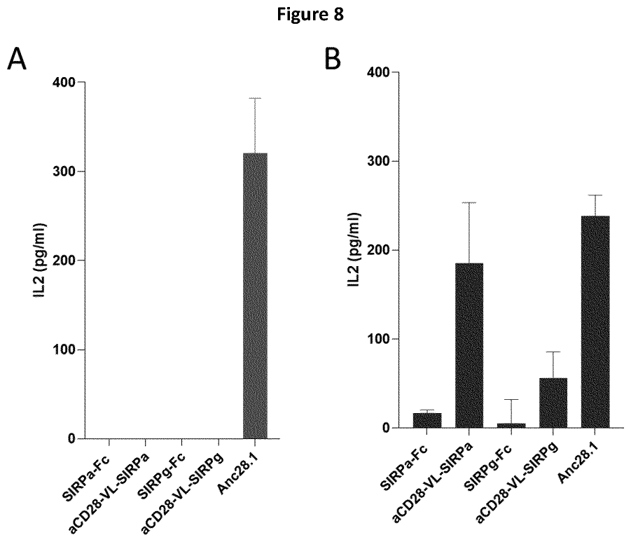

More particularly, when the dimeric Fc domain is a heterodimeric Fc domain, the bifunctional molecule comprises a first monomer comprising an antigen-binding domain covalently linked to the N-terminal end of the first heterodimeric Fc chain optionally via a peptide linker, said first heterodimeric Fc chain being covalently linked by its C-terminal end to an immune-stimulating moiety, optionally via a peptide linker, and a second monomer comprising a complementary second heterodimeric Fc chain devoid of antigen-binding domain and of immune-stimulating moiety. Optionally, said second monomer comprising a complementary second heterodimeric Fc chain is devoid of any other functional moiety. Still more particularly, the bifunctional molecule comprises a first monomer comprising an antigen-binding domain covalently linked via C-terminal end to N-terminal end of a first heterodimeric Fc chain optionally via a peptide linker, said first heterodimeric Fc chain being covalently linked by its C-terminal end to the N-terminal end of the immune-stimulating moiety, optionally via a peptide linker, and a second monomer comprising a complementary second heterodimeric Fc chain devoid of antigen-binding domain and of immune-stimulating moiety, preferably devoid of any other functional moiety. Such a bifunctional molecule is illustrated in FIG. 1, with SIRPγ as illustration of an immune-stimulating moiety.

Optionally, the single antigen-binding domain selected from the group consisting of a Fab, a Fab′, a scFV and a sdAb.

Accordingly, in one aspect, the bifunctional molecule according to the invention comprises or consists of:
(a) an antigen-binding domain that binds a target specifically expressed on immune cells surface, which comprises (i) one heavy chain with a first Fc chain, and (ii) one light chain,
(b) an immune-stimulating moiety, and
(c) a complementary second Fc chain,
   wherein the immune-stimulating moiety is covalently linked, optionally via a peptide linker, preferably by its N-terminal end, to the C-terminal end of the first Fc chain. The first Fc chain and the second Fc chain form together a dimeric Fc domain.

In a particular aspect, the bifunctional molecule comprises
one antibody heavy chain including VH-CH1-hinge-CH2-CH3 linked at its C-terminal end to an immune-stimulating moiety,
one antibody light chain including VL-CL (constant light chain), the VH-CH1 moiety and the VL-CL moiety forming together an antigen binding domain that binds a target specifically expressed on immune cells surface, and
one Fc chain comprising CH2-CH3, optionally hinge-CH2-CH3, forming with the CH2-CH3 of the antibody heavy chain a dimeric Fc domain.

According to an alternative aspect, when the dimeric Fc domain is a heterodimeric Fc domain, the bifunctional molecule comprises a first monomer comprising an antigen-binding domain covalently linked to the N-terminal end of the first heterodimeric Fc chain optionally via a peptide linker, a second monomer comprising a complementary second heterodimeric Fc chain devoid of antigen-binding domain and of immuno-stimulating cytokine, and said antigen-binding domain comprises a heavy variable chain and a light variable chain and the immune-stimulating moiety is linked, optionally via a peptide linker, at the C terminal end of the light chain of said antigen-binding domain. Optionally, said immune-stimulating moiety is linked, optionally via a peptide linker, at the C terminal end of the light chain of said antigen-binding domain by its N terminal end.

Accordingly, in this aspect, the bifunctional molecule according to the invention comprises or consists of:
(a) an antigen-binding domain that binds a target specifically expressed on immune cells surface, which comprises (i) one heavy chain with a first Fc chain, and (ii) one light chain,
(b) an immune-stimulating moiety, and
(c) a complementary second Fc chain,
   wherein the immune-stimulating moiety is covalently linked, optionally via a peptide linker, preferably by its N-terminal end, to the C-terminal end of the light chain. The first Fc chain and the second Fc chain form together a dimeric Fc domain.

In a particular aspect, the bifunctional molecule comprises
one antibody heavy chain including VH-CH1-hinge-CH2-CH3,
one antibody light chain including VL-CL (constant light chain) linked at its C-terminal end to an immune-stimulating moiety, the VH-CH1 moiety and the VL-CL moiety forming together an antigen binding domain that binds a target specifically expressed on immune cells surface, and
one Fc chain comprising CH2-CH3, optionally hinge-CH2-CH3, forming with the CH2-CH3 of the antibody heavy chain a dimeric Fc domain.

The immune-stimulating moiety, the antigen binding domain that binds a target specifically expressed on immune cells surface, the Fc domain and the optional linkers are as further defined below in any of the aspects.

### Immune-Stimulating Moiety

The immune-stimulating moiety is capable of stimulating or activating an immune cell. The immune cell can be selected in the non-exhaustive list comprising B cells, T cells, in particular CD4+ T cells and CD8+ T cells, NK cells, NKT cells, APC cells, dendritic cells and monocytes. In a preferred aspect, the immune cells are T cells, more specifically CD8+ T cells, effector T cells or exhausted T cells.

Preferably, the immune-stimulating moiety is selected from the group consisting of cytokines receptors, chemokines receptors, costimulatory molecules, inhibitory or coinhibitory molecules, enzymes, molecular chaperone inhibitors and human transmembrane immune protein of type I, or a fragment thereof, preferably the extracellular domain thereof. Preferably, such fragment retains the biological activity of the immune-stimulating moiety. Particularly, the immune-stimulating moiety has a size comprised between 10 kDa and 50 kDa. Preferably, the immune-stimulating moiety is a peptide, a polypeptide or a protein. In one aspect, the immune-stimulating moiety is a non-antibody entity or portion.

For instance, the immune-stimulating moiety can be selected from: T-cell growth factors, in particular growth factors to increase number and repertoire of naive T cells, growth factors to increase the number of dendritic cells (DCs), agonists to activate DCs and other antigen-presenting cells (APCs), adjuvants to allow and augment cancer vaccines, agonists to activate and stimulate T cells, inhibitors of T-cell checkpoint blockade, T-cell growth factors to increase the growth and survival of immune T cells, agents to inhibit, block, or neutralize cancer cell and immune cell-derived immunosuppressive cytokine. The immune-stimulating moiety can also consist in one or more other binding molecules, a receptor or an extracellular domain thereof, a ligand of a receptor or a fragment thereof having the same functional activity.

The immune-stimulating moiety may be mutated or altered so that the biological activity is altered, e.g. the biological activity is increased, decreased or totally inhibited.

In a specific aspect, the immune-stimulating moiety is a membrane protein or a fragment thereof, especially an extracellular fragment thereof, the protein being selected from the group consisting of SIRPγ, SIRPα, SIRPβ, CD80, CD86, LIGHT, CTLA-4, TIGIT, CD40L, OX40L, APRIL and GITRL or a variant thereof having at least 80, 85, 90, 91, 92, 93, 94, 95, 96, 97, 98 or 99% of identity with the wildtype protein or the extracellular fragment thereof or having 1 to 10 modifications selected from the group consisting of addition, deletion, substitution and combinations thereof.

In particular, the immune-stimulating moiety can be a protein selected in the list of Table D below, or a fragment thereof, especially an extracellular fragment thereof, or a variant thereof having at least 80, 85, 90, 91, 92, 93, 94, 95, 96, 97, 98 or 99% of identity with the wildtype protein or the extracellular fragment thereof or having 1 to 10 modifications selected from the group consisting of addition, deletion, substitution and combinations thereof.

TABLE D
List of immune-stimulating membrane proteins

| | | Uniprot |
|---|---|---|
| Name | Official name | reference |
| SIRPG | Signal-regulatory protein gamma, SIRP-gamma (CD172 antigen-like family | Q9P1W8 |
| | member B) (Signal-regulatory protein beta-2, SIRP-b2, SIRP-beta-2) (CD | |
| | antigen CD172g) | |
| SIRPa | Tyrosine-protein phosphatase non-receptor type substrate 1 (SHP | P78324 |
| | substrate 1, SHPS-1, Brain Ig-like molecule with tyrosine-based activation | |
| | motifs, Bit, CD172 antigen-like family member A, Inhibitory receptor SHPS- | |
| | 1, Macrophage fusion receptor, MyD-1 antigen, Signal-regulatory protein | |
| | alpha-1, Sirp-alpha-1, Signal-regulatory protein alpha-2, Sirp-alpha-2, | |
| | Signal-regulatory protein alpha-3, Sirp-alpha-3, p84, CD172a) | |
| SIRB1_{—} | Signal-regulatory protein beta-1, SIRP-beta-1 (CD172 antigen-like family | O00241 |
| | member B) (CD antigen CD172b) | |
| SIRB2_{—} | Signal-regulatory protein beta-2, SIRP-beta-2 (Protein tyrosine | Q5JXA9 |
| | phosphatase non-receptor type substrate 1-like 3) (Protein tyrosine | |
| | phosphatase non-receptor type substrate protein) | |
| CD80 | T-lymphocyte activation antigen CD80 (Activation B7-1 antigen, BB1, CTLA- | P33681 |
| | 4 counter-receptor B7.1, B7) | |
| CD86 | T-lymphocyte activation antigen CD86 (Activation B7-2 antigen) (B70) | P42081 |
| | (BU63) (CTLA-4 counter-receptor B7.2) (FUN-1) (CD antigen CD86) | |
| LIGHT | Tumor necrosis factor ligand superfamily member 14(Herpes virus entry | O43557 |
| | mediator ligand, HVEM-L, Herpesvirus entry mediator ligand) (CD antigen | |
| | CD258) [Cleaved into: Tumor necrosis factor ligand superfamily member | |
| | 14, membrane form; Tumor necrosis factor ligand superfamily member 14, | |
| | soluble form] | |
| CTLA-4 | Cytotoxic T-lymphocyte protein 4 (Cytotoxic T-lymphocyte-associated | P16410 |
| | antigen 4, CTLA-4) (CD antigen CD152) | |
| TIGIT | T-cell immunoreceptor with Ig and ITIM domains (V-set and | Q495A1 |
| | immunoglobulin domain-containing protein 9) (V-set and transmembrane | |
| | domain-containing protein 3) | |
| CD40L | CD40 ligand, CD40-L (T-cell antigen Gp39) (TNF-related activation protein, | P29965 |
| | TRAP) (Tumor necrosis factor ligand superfamily member 5) (CD antigen | |
| | CD154) | |
| OX40L | Tumor necrosis factor receptor superfamily member 4 (ACT35 antigen) | P43489 |
| | (OX40L receptor) (TAX transcriptionally-activated glycoprotein 1 receptor) | |
| | (CD antigen CD134) | |
| APRIL | Tumor necrosis factor ligand superfamily member 13 (A proliferation- | Q9D777 |
| | inducing ligand, APRIL) (CD antigen CD256) | |
| GITRL | Tumor necrosis factor ligand superfamily member 18 (GITR ligand, GITRL) | Q7TS55 |
| | (Glucocorticoid-induced TNF-related ligand) | |

Accordingly, the immune-stimulating moiety is selected from the group consisting of SIRPγ, SIRPα, SIRPβ, CD80, CD86, LIGHT, CTLA-4, TIGIT, CD40L, OX40L, APRIL and GITRL or a fragment thereof comprising the extracellular part thereof or a variant thereof having at least 80, 85, 90, 91, 92, 93, 94, 95, 96, 97, 98 or 99% of identity with the wildtype protein or the extracellular fragment thereof or having 1 to 10 modifications selected from the group consisting of addition, deletion, substitution and combinations thereof.

In a particularly, the immune-stimulating moiety is selected from the group consisting of SIRPγ and SIRPα or a fragment thereof comprising the extracellular part thereof or a variant thereof having at least 80, 85, 90, 91, 92, 93, 94, 95, 96, 97, 98 or 99% of identity with the wildtype protein or the extracellular fragment thereof or having 1 to 10 modifications selected from the group consisting of addition, deletion, substitution and combinations thereof.

### SIRPα, SIRPγ and Fragments and Variants Thereof

In a particular aspect, the immune-stimulating moiety is a human SIRPα or fragments and variants thereof. The typical wild-type SIRPα human protein of about 504 amino acids, preferably the extracellular domain of the wild-type human SIRPα protein (e.g. consisting of amino acids from positions 31 to 373 of the wild-type human SIRPα). The SIRPα fragment used as immune-stimulating moiety is preferably devoid of its transmembrane domain and/or cytoplasmic domain. For instance, the SIRPα fragment may consist of its extracellular domain, even more preferably consists essentially of the 31 to 373 amino acids of the wild-type human SIRPα, in particular SEQ ID NO: 1.

The most common human SIRPα variants are SIRPα v1 and SIRPα v2 (accession number NP_542970 (P78324) and CAA71403). The SIRPα family may be divided into these two subsets; namely the SIRPα v1 isoform family and the SIRPα v2 isoform family. These families include the SIRPα Isoform 2 (identifier: P78324-2) and the SIRPα Isoform 4 (identifier: P78324-4), respectively. In one aspect, the SIRPα variant is selected from the group consisting of the SIRPα isoform 2 (P78324-2) and the SIRPα isoform 4 (P78324-4).

In a particular aspect, the extracellular part of SIRPα has a sequence as shown in SEQID NO: 1 or a sequence having at least 80, 85, 90, 91, 92, 93, 94, 95, 96, 97, 98 or 99% of identity with SEQ ID NO: 1 or having 1 to 10 modifications selected from the group consisting of addition, deletion, substitution and combinations thereof with respect to the sequence of SEQ ID NO: 1.

In another particular aspect, the immune stimulating moiety is SIRPγ, a fragment thereof, especially the extracellular part thereof, or a variant of SIRPγ or a fragment thereof.

SIRPγ has similar extracellular structure than SIRPα but different cytoplasmic regions giving contrasting types of signals. Indeed, SIRPα and SIRPγ comprises 3 Ig-like extracellular domains: an Ig-like V-type, encoded by amino acids 32-137 (domain D1), an Ig-like C1-type 1 encoded by amino acids at positions 148-247 (domain D2), Ig-like C1-type 2 encoded by amino acids at positions 254-348 (domain D3).

In a particular aspect, the extracellular part of SIRPγ has a sequence as shown in SEQID NO: 2 or a sequence having at least 80, 85, 90, 91, 92, 93, 94, 95, 96, 97, 98 or 99% of identity with SEQ ID NO: 2 or having 1 to 10 modifications selected from the group consisting of addition, deletion, substitution and combinations thereof with respect to the sequence of SEQ ID NO: 2.

Then, in one aspect, the SIRPα or SIRPγ variant may comprise i) D1 domain of SIRPγ, D2 and D3 domains of SIRPα ii) D1 and D2 domains of SIRPγ and D3 domain of SIRPα iii) D1 domain of SIRPγ, D2 domain of SIRPα and D3 domain of SIRPγ, iv) D1 domain of SIRPα, D2 and D3 domains of SIRPγ, v) D1 and D2 domains of SIRPα and D3 domain of SIRPg or vi) D1 domain of SIRPα, D2 domain of SIRPγ and D3 domain of SIRPα.

In a particular aspect, the SIRPα or SIRPγ consists of truncations or fragment of the extracellular domain of SIRPα or SIRPγ, specifically comprising or consisting of binding regions or of the amino acids within the set of contact residues that interact with CD47.

In one aspect, the affinity of SIRPα or SIRPγ protein can be measured using in vitro assays. Preferably, the SIRPα or SIRPγ variants according to the invention maintain the affinity to CD47 of at least 10%, 20%, 30%, 40%, 50%, 60% in comparison with the wild type human SIRPα or SIRPγ, respectively, preferably at least 80%, 90%, 95% and even more preferably 99% in comparison with the wild type SIRPα or SIRPγ, respectively.

### Antigen Binding Domain Specifically Targeting Immune Cells

According to the invention, the antigen binding domain specifically binds to a target expressed on immune cells surface, particularly targets that are only or specifically expressed on immune cells. In particular, the antigen binding domain is not directed towards a target expressed on tumoral cells.

With regard to the “binding” capacity of the antigen binding domain, the terms “bind” or “binding” refer to antibodies including antibody fragments and derivatives that recognize and contact another peptide, polypeptide, protein or molecule. The terms “specific binding”, “specifically binds to,” “specific for,” “selectively binds” and “selective for” a particular target mean that the antigen binding domain recognizes and binds a specific target, but does not substantially recognize or bind other molecules in a sample. For example, an antibody that specifically (or preferentially) binds to an antigen is an antibody that binds the antigen for example with greater affinity, avidity, more readily, and/or with greater duration than it binds to other molecules. Preferably, the term “specific binding” means the contact between an antibody and an antigen with a binding affinity equal or lower than 10⁻⁷ M. In certain aspects, antibodies bind with affinities equal or lower than 10⁻⁸ M, 10⁻⁹ M or 10⁻¹⁰ M.

Optionally, the antigen-binding domain can be a Fab domain, a Fab′, a single-chain variable fragment (scFV) or a single domain antibody (sdAb). The antigen-binding domain preferably comprises a heavy chain variable region (VH) and a light chain variable region (VL).

When the antigen-binding domain is a Fab or a Fab′, the bifunctional molecule comprises one heavy chain and one light chain constant domain (i.e. CH and CL), the heavy chain being linked at its C-terminal end to the immune-stimulating moiety.

As used herein, the term “target” refers to a carbohydrate, lipid, peptide, polypeptide, protein, antigen or epitope that is specifically recognized or targeted by the antigen binding domain according to the invention and expressed on the external surface of immune cells. With regards to the expression of a target on the surface of immune cells, the term “expressed” refers to a target, such as carbohydrates, lipids, peptides, polypeptides, proteins, antigens or epitopes that are present or presented at the outer surface of a cell. The term “specifically expressed” mean that the target is expressed on immune cells, but is not substantially expressed by other cell type, particularly such as tumoral cells.

In one aspect, the target is specifically expressed by immune cells in a healthy subject or in a subject suffering from a disease, in particular such as a cancer. This means that the target has a higher expression level in immune cells than in other cells or that the ratio of immune cells expressing the target by the total immune cells is higher than the ratio of other cells expressing the target by the total other cells. Preferably the expression level or ratio is higher by a factor 2, 5, 10, 20, 50 or 100. More specifically, it can be determined for a particular type of immune cells, for instance T cells, more specifically CD8+ T cells, effector T cells or exhausted T cells, or in a particular context, for instance a subject suffering of a disease such as a cancer or an infection.

“Immune cells” as used herein refers to cells involved in innate and adaptive immunity for example such as white blood cells (leukocytes) which are derived from hematopoietic stem cells (HSC) produced in the bone marrow, lymphocytes (T cells, B cells, natural killer (NK) cells and Natural Killer T cells (NKT)) and myeloid-derived cells (neutrophil, eosinophil, basophil, monocyte, macrophage, dendritic cells). In particular, the immune cell can be selected in the non-exhaustive list comprising B cells, T cells, in particular CD4⁺ T cells and CD8⁺ T cells, NK cells, NKT cells, APC cells, macrophages, dendritic cells and monocytes.

Preferably, the antigen binding domain specifically binds to a target expressed immune cells selected from the group consisting of B-cells, T-cells, Natural killer, dendritic cells, monocytes and innate lymphoid cells (ILCs).

Even more preferably, the immune cell is a T cell. “T cell” or “T lymphocytes” as used herein includes for example CD4+ T cells, CD8+ T cells, T helper 1 type T cells, T helper 2 type T cells, T regulator, T helper 17 type T cells and inhibitory T cells. In a very particular aspect, the immune cell is an exhausted T cell.

The target can be a receptor expressed at the surface of the immune cells, especially T cells. The receptor can be an inhibitor receptor. Alternatively, the receptor can be an activating receptor.

In one aspect, the target is selected from the group consisting PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, 87-1, 284, DR3, CD101, CD44, SIRPG, CD28H, CD38, CD3, PDL1; PDL2, and PDL1. Such targets are more particularly described in the Table F below.

TABLE E
Example of target of interest.

| Name | Official name | Uniprot reference |
|---|---|---|
| 2B4 | Natural killer cell receptor 2B4 (NK cell type I receptor protein 2B4, | Q07763 |
| | NKR2B4) (Non-MHC restricted killing associated) (SLAM family | |
| | member 4, SLAMF4) (Signaling lymphocytic activation molecule 4) | |
| | (CD antigen CD244) | |
| 4-1BB | Tumor necrosis factor receptor superfamily member 9 (4-1BB ligand | Q07011 |
| | receptor, CD137) | |
| BTLA | B- and T-lymphocyte attenuator (B- and T-lymphocyte-associated | Q7Z6A9 |
| | protein) (CD antigen CD272) | |
| CD101 | Immunoglobulin superfamily member 2, IgSF2 (Cell surface | Q93033 |
| | glycoprotein V7) (Glu-Trp-Ile EWI motif-containing protein 101, EWI- | |
| | 101) (CD antigen CD101) | |
| CD160 | CD160 antigen (Natural killer cell receptor BY55) | O95971 |
| CD27 | CD27 antigen (CD27L receptor) (T-cell activation antigen CD27) (T14) | P26842 |
| | (Tumor necrosis factor receptor superfamily member 7) (CD antigen | |
| | CD27) | |
| CD28 | T-cell-specific surface glycoprotein CD28 (TP44) | P10747 |
| CD28H | Transmembrane and immunoglobulin domain-containing protein 2 | Q96BF3 |
| | (CD28 homolog) (Immunoglobulin and proline-rich receptor 1, IGPR- | |
| | 1) | |
| CD3 | T-cell surface glycoprotein CD3 | P07766 (CD3e) |
| | | P04234 (CD3d) |
| | | P09693 (CD3g) |
| CD30 | Tumor necrosis factor ligand superfamily member 8 (CD30 ligand, | P32971 |
| | CD30-L) (CD antigen CD153) | |
| CD38 | ADP-ribosyl cyclase/cyclic ADP-ribose hydrolase 1 (ADPRC 1, cADPr | P28907 |
| | hydrolase 1) | |
| CD39 | Ectonucleoside triphosphate diphosphohydrolase-1 (NTPDase 1, | P49961 |
| | Ecto-apyrase, ATPDase 1, or Lymphoid cell activation antigen) | |
| CD40L | CD40 ligand (T-cell antigen Gp39, TNF-related activation protein, | P29965 |
| | Tumor necrosis factor ligand superfamily member 5, CD154) | |
| CD44 | CD44 antigen (Epican, Extracellular matrix receptor III, GP90 | P16070 |
| | lymphocyte homing/adhesion receptor, HUTCH-I, Heparan sulfate | |
| | proteoglycan, Hermes antigen, Hyaluronate receptor, Phagocytic | |
| | glycoprotein 1, Phagocytic glycoprotein I) | |
| CTLA-4 | Cytotoxic T-lymphocyte protein 4 (Cytotoxic T-lymphocyte-associated | P16410 |
| | antigen 4, CTLA-4) (CD antigen CD152) | |
| DR3 | Death receptor 3 (Tumor necrosis factor receptor superfamily | Q93038 |
| | member 25, WSL, Apo-3, LARD) | |
| GITR | Tumor necrosis factor receptor superfamily member 18 (Activation- | Q9Y5U5 |
| | inducible TNFR family receptor, Glucocorticoid-induced TNFR-related | |
| | protein, CD357) | |
| HVEM | Tumor necrosis factor receptor superfamily member 14 (Herpes virus | Q92956 |
| | entry mediator A, Herpesvirus entry mediator A, HveA) (Tumor | |
| | necrosis factor receptor-like 2, TR2) (CD antigen CD270) | |
| ICOS | Inducible T-cell costimulator (Activation-inducible lymphocyte | Q9Y6W8 |
| | immunomediatory molecule, CD278) | |
| LAG3 | Lymphocyte activation gene 3 protein, LAG-3 (Protein FDC) (CD | P18627 |
| | antigen CD223) | |
| LFA-1 | Leukocyte adhesion glycoprotein LFA-1 alpha chain (Integrin alpha-L, | P20701 |
| | CD11 antigen-like family member A) | |
| NKG2D | NKG2-D type II integral membrane protein (Killer cell lectin-like | P26718 |
| | receptor subfamily K member 1, NK cell receptor D, NKG2-D- | |
| | activating NK receptor, CD314) | |
| OX40 | Tumor necrosis factor receptor superfamily member 4 (ACT35 | P43489 |
| | antigen, AX transcriptionally-activated glycoprotein 1 receptor) | |
| PD-1 | Programmed cell death protein 1 (CD279) | Q15116 |
| PDL1 | Programmed cell death 1 ligand 1, PDL1, PD-L1, PDCD1 ligand 1, B7- | Q9NZQ7 |
| | H1, CD274 | |
| PDL2 | Programmed cell death 1 ligand 2, PD-1 ligand 2, PD-L2, PDCD1 ligand | Q9BQ51 |
| | 2, Programmed death ligand 2 (Butyrophilin B7-DC, B7-DC) (CD | |
| | antigen CD273) | |
| SIRPG | Signal-regulatory protein gamma, SIRP-gamma (CD172 antigen-like | Q9P1W8 |
| | family member B) (Signal-regulatory protein beta-2, SIRP-b2, SIRP- | |
| | beta-2) (CD antigen CD172g) | |
| TIGIT | T-cell immunoreceptor with Ig and ITIM domains (V-set and | Q495A1 |
| | immunoglobulin domain-containing protein 9) (V-set and | |
| | transmembrane domain-containing protein 3) | |
| Tim-1 | Hepatitis A virus cellular receptor 1 (T-cell immunoglobulin and | Q96D42 |
| | mucin domain-containing protein 1, Kidney injury molecule 1, KIM-1, | |
| | T-cell immunoglobulin mucin receptor 1, T-cell membrane protein 1, | |
| | CD365) | |
| TIM3 | Hepatitis A virus cellular receptor 2, HAVcr-2 (T-cell immunoglobulin | Q8TDQ0 |
| | and mucin domain-containing protein 3, TIMD-3) (T-cell | |
| | immunoglobulin mucin receptor 3, TIM-3) (T-cell membrane protein | |
| | 3) | |

Then, in this aspect, the antigen binding domain specifically binds a target selected from the group consisting PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, 87-1, 284, DR3, CD101, CD44, SIRPG, CD28H, CD38, CD3, PDL2 and PDL1.

In a particular aspect, the immune cell is an exhausted T cell and the target of the antigen binding domain is a factor expressed on the surface of exhausted T cells. T cell exhaustion is a state of T cell progressive loss of function, proliferation capacity and cytotoxic potential, eventually leading to their deletion. T cell exhaustion can be triggered by several factors such as persistent antigen exposure or inhibitory receptors including PD-1, TIM3, CD244, CTLA-4, LAG-3, BTLA, TIGIT and CD160. Preferably, such exhaustion factor is selected from the group consisting of PD-1, TIM3, CD244, CTLA-4, LAG3, BTLA, TIGIT and CD160.

In a preferred aspect, the antigen binding domain has an antagonist activity on the target.

Numerous antibodies directed against PD-1, CD28, TIM3, CD244, CTLA-4, LAG-3, BTLA, TIGIT and CD160 have already been described in the art.

Several anti-PD-1 are already clinically approved, and others are still in clinical developments. For instance, the anti-PD1 antibody can be selected from the group consisting of Pembrolizumab (also known as Keytruda lambrolizumab, MK-3475), Nivolumab (Opdivo, MDX-1106, BMS-936558, ONO-4538), Pidilizumab (CT-011), Cemiplimab (Libtayo), Camrelizumab, AUNP12, AMP-224, IsBGB-A317 (Tisleizumab), PDR001 (spartalizumab), MK-3477, PF-06801591, JNJ-63723283, genolimzumab (CBT-501), LZM-009, BCD-100, SHR-1201, BAT-1306, AK-103 (HX-008), MEDI-0680 (also known as AMP-514) MEDI0608, JS001 (see Si-Yang Liu et al., J. Hematol. Oncol.10:136 (2017)), BI-754091, TSR-042 (also known as ANB011), GLS-010 (also known as WBP3055), AM-0001 (Armo), STI-1110 (see WO 2014/194302), AGEN2034 (see WO 2017/040790), MGA012 (see WO 2017/19846), or IBI308 (see WO 2017/024465, WO 2017/025016, WO 2017/132825, and WO 2017/133540), monoclonal antibodies 5C4, 17D8, 2D3, 4H1, 4A11, 7D3, and 5F4, described in WO 2006/121168. Bifunctional or bispecific molecules targeting PD-1 are also known such as RG7769 (Roche), XmAb20717 (Xencor), MEDI5752 (AstraZeneca), FS118 (F-star), SL-279252 (Takeda) and XmAb23104 (Xencor).

In a particular aspect, the anti-PD1 antibody can be Pembrolizumab (also known as Keytruda lambrolizumab, MK-3475) or Nivolumab (Opdivo, MDX-1106, BMS-936558, ONO-4538).

Antibodies directed against TIM3 and bifunctional or bispecific molecules targeting TIM3 are also known such as Sym023, TSR-022, MBG453, LY3321367, INCAGN02390, BGTB-A425, LY3321367, RG7769 (Roche). In some aspects, a TFM-3 antibody is as disclosed in International Patent Application Publication Nos. WO2013006490, WO2016/161270, WO 2018/085469, or WO 2018/129553, WO 2011/155607, U.S. Pat. No. 8,552,156, EP 2581113 and U.S 2014/044728.

Antibodies directed against CTLA-4 and bifunctional or bispecific molecules targeting CTLA-4 are also known such as ipilimumab, tremelimumab, MK-1308, AGEN-1884, XmAb20717 (Xencor), MEDI5752 (AstraZeneca). Anti-CTLA-4 antibodies are also disclosed in WO18025178, WO19179388, WO19179391, WO19174603, WO19148444, WO19120232, WO19056281, WO19023482, WO18209701, WO18165895, WO18160536, WO18156250, WO18106862, WO18106864, WO18068182, WO18035710, WO18025178, WO17194265, WO17106372, WO17084078, WO17087588, WO16196237, WO16130898, WO16015675, WO12120125, WO09100140 and WO07008463.

Antibodies directed against LAG3 and bifunctional or bispecific molecules targeting LAG-3 are also known such as BMS- 986016, IMP701, MGD012 or MGD013 (bispecific PD-1 and LAG-3 antibody). Anti-LAG-3 antibodies are also disclosed in WO2008132601, EP2320940, WO19152574.

Antibodies directed against BTLA are also known in the art such as hu Mab8D5, hu Mab8A3, hu Mab21H6, hu Mab19A7, or hu Mab4C7. The antibody TAB004 against BTLA are currently under clinical trial in subjects with advanced malignancies. Anti-BTLA antibodies are also disclosed in WO08076560, WO10106051 (e.g., BTLA8.2), WO11014438 (e.g., 4C7), WO17096017 and WO17144668 (e.g., 629.3).

Antibodies directed against TIGIT are also known in the art, such as BMS-986207 or AB154, BMS-986207 CPA.9.086, CHA.9.547.18, CPA.9.018, CPA.9.027, CPA.9.049, CPA.9.057, CPA.9.059, CPA.9.083, CPA.9.089, CPA.9.093, CPA.9.101, CPA.9.103, CHA.9.536.1, CHA.9.536.3, CHA.9.536.4, CHA.9.536.5, CHA.9.536.6, CHA.9.536.7, CHA.9.536.8, CHA.9.560.1, CHA.9.560.3, CHA.9.560.4, CHA.9.560.5, CHA.9.560.6, CHA.9.560.7, CHA.9.560.8, CHA.9.546.1, CHA.9.547.1, CHA.9.547.2, CHA.9.547.3, CHA.9.547.4, CHA.9.547.6, CHA.9.547.7, CHA.9.547.8, CHA.9.547.9, CHA.9.547.13, CHA.9.541.1, CHA.9.541.3, CHA.9.541.4, CHA.9.541.5, CHA.9.541.6, CHA.9.541.7, and CHA.9.541.8 as disclosed in WO19232484. Anti-TIGIT antibodies are also disclosed in WO16028656, WO16106302, WO16191643, WO17030823, WO17037707, WO17053748, WO17152088, WO18033798, WO18102536, WO18102746, WO18160704, WO18200430, WO18204363, WO19023504, WO19062832, WO19129221, WO19129261, WO19137548, WO19152574, WO19154415, WO19168382 and WO19215728.

Antibodies directed against CD160 are also known in the art, such as CL1-R2 CNCM I-3204 as disclosed in WO06015886, or others as disclosed in WO10006071, WO10084158, WO18077926.

Antibodies directed against PD-L1 are also known in the art. Examples of monoclonal antibodies that bind to human PD-L1, and useful for the present invention, are described in WO 2007/005874, WO 2010/036959, WO 2010/077634, WO 2010/089411, WO 2013/019906, WO 2013/079174, WO 2014/100079, WO 2015/061668, and U.S. Pat. No. 8,552,154, U.S. Pat. No. 8,779,108 and U.S. Pat. No. 8,383,796. Specific anti-human PD-L1 monoclonal antibodies include, for example without limitation, avelumab (MSB0010718C), durvalumab (MEDI4736, an engineered IgG1 kappa monoclonal antibody with triple mutations in the Fc domain to remove ADCC), atezolizumab (MPLDL3280A), MPDL3280A (an IgG1 -engineered anti-PD-L1 antibody), and BMS-936559 (a fully human, anti-PD-L1, IgG4 monoclonal antibody).

Antibodies directed against CD28 are also known in the art, such as Theralizumab (TGN1412) as disclosed in WO2006/050949, WO2002/051871 or in Nunes et al. International immunology vol. 5,3 (1993): 311-5. Particularly, antibodies directed against CD28 can be Theralizumab (TGN1412), TGN1112, CD28.1 to CD28.6 as disclosed in Nunes et al. 1993, Anc28.1 as disclosed in Waibler et al. PLOS ONE (2008) 3(3): e1708., or anti-CD28 antibodies as disclosed in EP3941941, EP3810282, EP3898695, EP3897715 or WO2022/061098.

In a preferred aspect, the antigen binding domain of the bifunctional molecule is an antibody, a fragment or a derivative thereof that is specific to PD-1, CD28, CTLA-4, BTLA, TIGIT, LAG3 and TIM3.

In another particular aspect, the target is PD-1 and the antigen binding domain of the bifunctional molecule is an antibody, a fragment or a derivative thereof or an antibody mimic that is specific to PD-1. Then, in a particular aspect, the antigen binding domain comprised in the bifunctional molecule according to the invention is an anti-PD1 antibody or antigen binding fragment thereof, preferably a human, humanized or chimeric anti-PD1 antibody or antigen binding fragment thereof. Preferably, the antigen binding domain is an antagonist of PD-1.

In another particular aspect, the target is CTLA-4 and the antigen binding domain of the bifunctional molecule is an antibody, a fragment or a derivative thereof or an antibody mimic that is specific to CTLA-4. Then, in a particular aspect, the antigen binding domain comprised in the bifunctional molecule according to the invention is an anti-CTLA-4 antibody or antigen binding fragment thereof, preferably a human, humanized or chimeric anti-CTLA-4 antibody or antigen binding fragment thereof. Preferably, the antigen binding domain is an antagonist of CTLA-4.

In another particular aspect, the target is BTLA and the antigen binding domain of the bifunctional molecule is an antibody, a fragment or a derivative thereof or an antibody mimic that is specific to BTLA. Then, in a particular aspect, the antigen binding domain comprised in the bifunctional molecule according to the invention is an anti-BTLA antibody or antigen binding fragment thereof, preferably a human, humanized or chimeric anti-BTLA antibody or antigen binding fragment thereof. Preferably, the antigen binding domain is an antagonist of BTLA.

In another particular aspect, the target is TIGIT and the antigen binding domain of the bifunctional molecule is an antibody, a fragment or a derivative thereof or an antibody mimic that is specific to TIGIT. Then, in a particular aspect, the antigen binding domain comprised in the bifunctional molecule according to the invention is an anti-TIGIT antibody or antigen binding fragment thereof, preferably a human, humanized or chimeric anti-TIGIT antibody or antigen binding fragment thereof. Preferably, the antigen binding domain is an antagonist of TIGIT.

In another particular aspect, the target is LAG-3 and the antigen binding domain of the bifunctional molecule is an antibody, a fragment or a derivative thereof or an antibody mimic that is specific to LAG-3. Then, in a particular aspect, the antigen binding domain comprised in the bifunctional molecule according to the invention is an anti-LAG-3 antibody or antigen binding fragment thereof, preferably a human, humanized or chimeric anti-LAG-3 antibody or antigen binding fragment thereof. Preferably, the antigen binding domain is an antagonist of LAG-3.

In another particular aspect, the target is TIM3 and the antigen binding domain of the bifunctional molecule is an antibody, a fragment or a derivative thereof or an antibody mimic that is specific to TIM3.

Then, in a particular aspect, the antigen binding domain comprised in the bifunctional molecule according to the invention is an anti-TIM3 antibody or antigen binding fragment thereof, preferably a human, humanized or chimeric anti-TIM3 antibody or antigen binding fragment thereof. Preferably, the antigen binding domain is an antagonist of TIM3.

In another particular aspect, the target is CD28 and the antigen binding domain of the bifunctional molecule is an antibody, a fragment or a derivative thereof or an antibody mimic that is specific to CD28. Then, in a particular aspect, the antigen binding domain comprised in the bifunctional molecule according to the invention is an anti-CD28 antibody or antigen binding fragment thereof, preferably a human, humanized or chimeric anti-CD28 antibody or antigen binding fragment thereof. Preferably, the antigen binding domain is an antagonist of CD28.

In a very specific aspect of the present disclosure, the antigen binding domain targets PD-1 and is derived from the antibody disclosed in WO2020/127366, the disclosure thereof being incorporated herein by reference.

Then, the antigen-binding domain comprises:
(i) a heavy chain variable domain comprising HCDR1, HCDR2 and HCDR3, and
(ii) a light chain variable domain comprising LCDR1, LCDR2 and LCDR3,
   wherein:
the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
the heavy chain CDR3 (HCDR3) comprises or consists of an amino acid sequence of SEQ ID NO: 54 wherein X1 is D or E and X2 is selected from the group consisting of T, H, A, Y, N, E and S, preferably in the group consisting of H, A, Y, N, E; optionally with one, two or three modification(s) selected from substitution(s), addition(s), deletion(s) and any combination thereof at any position but positions 2, 3, 7 and 8 of SEQ ID NO: 54;
the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
the light chain CDR2 (LCDR2) comprises or consists of an amino acid sequence of SEQ ID NO: 66, optionally with one, two or three modification(s) selected from substitution(s), addition(s), deletion(s) and any combination thereof; and
the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In one aspect, the antigen-binding domain comprises:
(i) a heavy chain variable domain comprising HCDR1, HCDR2 and HCDR3, and
(ii) a light chain variable domain comprising LCDR1, LCDR2 and LCDR3,
   wherein:
the heavy chain CDR1 (HCDR1) comprises or consists of an amino acid sequence of SEQ ID NO: 51, optionally with one, two or three modification(s) selected from substitution(s), addition(s), deletion(s) and any combination thereof at any position but position 3 of SEQ ID NO: 51;
the heavy chain CDR2 (HCDR2) comprises or consists of an amino acid sequence of SEQ ID NO: 53, optionally with one, two or three modification(s) selected from substitution(s), addition(s), deletion(s) and any combination thereof at any position but positions 13, 14 and 16 of SEQ ID NO: 53;
the heavy chain CDR3 (HCDR3) comprises or consists of an amino acid sequence of SEQ ID NO: 54 wherein either X1 is D and X2 is selected from the group consisting of T, H, A, Y, N, E, and S preferably in the group consisting of H, A, Y, N, E; or X1 is E and X2 is selected from the group consisting of T, H, A, Y, N, E and S, preferably in the group consisting of H, A, Y, N, E and S; optionally with one, two or three modification(s) selected from substitution(s), addition(s), deletion(s) and any combination thereof at any position but positions 2, 3, 7 and 8 of SEQ ID NO: 54;
the light chain CDR1 (LCDR1) comprises or consists of an amino acid sequence of SEQ ID NO: 63 wherein X is G or T, optionally with one, two or three modification(s) selected from substitution(s), addition(s), deletion(s) and any combination thereof at any position but positions 5, 6, 10, 11 and 16 of SEQ ID NO: 63;
the light chain CDR2 (LCDR2) comprises or consists of an amino acid sequence of SEQ ID NO: 66, optionally with one, two or three modification(s) selected from substitution(s), addition(s), deletion(s) and any combination thereof; and
the light chain CDR3 (LCDR3) comprises or consists of an amino acid sequence of SEQ ID NO: 16, optionally with one, two or three modification(s) selected from substitution(s), addition(s), deletion(s) and any combination thereof at any position but positions 1, 4 and 6 of SEQ ID NO: 16.

In another embodiment, the antigen-binding domain comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or SEQ ID NO: 65, a CDR2 of SEQ ID NO: 66and a CDR3 of SEQ ID NO: 16.

In another aspect, the antigen-binding domain comprises or consists essentially of:
(i) a heavy chain comprising a CDR1 of SEQ ID NO: 51, a CDR2 of SEQ ID NO: 53 and a CDR3 of SEQ ID NO: 55; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 56; and (ii) a light chain comprising a CDR1 of SEQ ID NO: 64, a CDR2 of SEQ ID NO: 66 and a CDR3 of SEQ ID NO: 16, or
(i) a heavy chain comprising a CDR1 of SEQ ID NO: 51, a CDR2 of SEQ ID NO: 53 and a CDR3 of SEQ ID NO: 57; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 58; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53 and a CDR3 of SEQ ID NO: 59; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 60; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 61; and (ii) a light chain comprising a CDR1 of SEQ ID NO: 64, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 62; and (ii) a light chain comprising a CDR1 of SEQ ID NO: 64, a CDR2 of SEQ ID NO: 66 and a CDR3 of SEQ ID NO: 16, or
(i) a heavy chain comprising a CDR1 of SEQ ID NO: 51, a CDR2 of SEQ ID NO: 53and a CDR3 of SEQ ID NO: 55; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 56; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 57; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 58; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 59; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 60; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 61; and (ii) a light chain comprising a CDR1 of SEQ ID NO: 65, a CDR2 of SEQ ID NO: 66 and a CDR3 of SEQ ID NO: 16; or
(i) a heavy chain comprising a CDR1 of SEQ ID NO: 51, a CDR2 of SEQ ID NO: 53and a CDR3 of SEQ ID NO: 62; and (ii) a light chain comprising a CDR1 of SEQ ID NO: 65, a CDR2 of SEQ ID NO: 66 and a CDR3 of SEQ ID NO: 16.

In an aspect, the antigen-binding domain comprises or consists essentially of:
(a) a heavy chain variable region (VH) comprising or consisting of an amino acid sequence of SEQ ID NO: 17, wherein X1 is D or E and X2 is selected from the group consisting of T, H, A, Y, N, E and S preferably in the group consisting of H, A, Y, N, E; optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 17;
(b) a light chain variable region (VL) comprising or consisting of an amino acid sequence of SEQ ID NO: 26, wherein X is G or T, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 26.

In another aspect, the antigen-binding domain comprises or consists essentially of:
(a) a heavy chain variable region (VH) comprising or consisting of an amino acid sequence of SEQ ID NO:18, 19, 20, 21, 22, 23, 24 or 25, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 18, 19, 20, 21, 22, 23, 24 or 25 respectively;
(b) a light chain variable region (VL) comprising or consisting of an amino acid sequence of SEQ ID NO: 27 or SEQ ID NO: 28, optionally with one, two or three modification(s) selected from substitution(s), addition(s), deletion(s) and any combination thereof at any position positions 3, 4, 7, 14, 17, 18, 28, 29, 33, 34, 39, 42, 44, 50, 81, 88, 94, 97, 99 and 105 of SEQ ID NO: 27 or SEQ ID NO: 28.

In another aspect, the antigen-binding domain comprises or consists essentially of:
(a) a heavy chain variable region (VH) comprising or consisting of an amino acid sequence of SEQ ID NO: 18, 19, 20, 21, 22, 23, 24 or 25;
(b) a light chain variable region (VL) comprising or consisting of an amino acid sequence of SEQ ID NO: 27 or SEQ ID NO: 28.

In one aspect, the bifunctional molecule comprises framework regions, in particular heavy chain variable region framework regions (HFR) HFR1, HFR2, HFR3 and HFR4 and light chain variable region framework regions (LFR) LFR1, LFR2, LFR3 and LFR4, especially HFR1, HFR2, HFR3 and HFR4 comprising an amino acid sequence of SEQ ID NOs: 41, 42, 43 and 44, respectively, optionally with one, two or three modification(s) selected from substitution(s), addition(s), deletion(s) and any combination thereof at any position but positions 27, 29 and 32 of HFR3, i.e., of SEQ ID NO: 43. Preferably, the bifunctional molecule comprises HFR1 of SEQ ID NO: 41, HFR2 of SEQ ID NO: 42, HFR3 of SEQ ID NO: 43 and HFR4 of SEQ ID NO: 44. In addition, the bifunctional molecule may comprise light chain variable region framework regions (LFR) LFR1, LFR2, LFR3 and LFR4 comprising an amino acid sequence of SEQ ID NOs: 45, 46, 47 and 48, respectively, optionally with one, two or three modification(s) selected from substitution(s), addition(s), deletion(s) and any combination thereof. Preferably, the bifunctional molecule comprises LFR1 of SEQ ID NO: 45, LFR2 of SEQ ID NO: 46, LFR3 of SEQ ID NO: 47 and LFR4 of SEQ ID NO: 48.

In another aspect, the antigen-binding domain comprises or consists essentially of any of the following combinations of a heavy chain variable region (VH) and a light chain variable region (VL):

| VH (SEQ ID NO:), optionally with one, two or | |
|---|---|
| three modification(s) selected from | VL (SEQ ID NO:), optionally with one, two or |
| substitution(s), addition(s), deletion(s) and any | three modification(s) selected from |
| combination thereof at any position but | substitution(s), addition(s), deletion(s) and any |
| positions 7, 16, 17, 20, 33, 38, 43, 46, 62, 63, 65, | combination thereof at any position positions 3, |
| 69, 73, 76, 78, 80, 84, 85, 88, 93, 95, 96, 97, 98, | 4, 7, 14, 17, 18, 28, 29, 33, 34, 39, 42, 44, 50, 81, |
| 100, 101, 105, 106 and 112 of SEQ ID NO: | 88, 94, 97, 99 and 105 of SEQ ID NO: |
| 18 | 27 |
| 18 | 28 |
| 19 | 27 |
| 19 | 28 |
| 20 | 27 |
| 20 | 28 |
| 21 | 27 |
| 21 | 28 |
| 22 | 27 |
| 22 | 28 |
| 23 | 27 |
| 23 | 28 |
| 24 | 27 |
| 24 | 28 |
| 25 | 27 |
| 25 | 28 |

In very particular aspect, the antigen-binding domain comprises or consists essentially of a heavy chain variable region (VH) of SEQ ID NO: 24 and a light chain variable region (VL) of SEQ ID NO: 28.

In a very specific aspect of the present disclosure, the antigen binding domain targets CD28 and is derived from the antibody disclosed in WO2006/050949, the disclosure thereof being incorporated herein by reference.

Then, the antigen-binding domain comprises:
(i) a heavy chain variable domain comprising HCDR1, HCDR2 and HCDR3, and
(ii) a light chain variable domain comprising LCDR1, LCDR2 and LCDR3,
   wherein:
the heavy chain CDR1 (HCDR1) comprises or consists of an amino acid sequence of SEQ ID NO: 77; optionally with one, two or three modification(s) selected from substitution(s), addition(s), deletion(s) and any combination thereof;
the heavy chain CDR2 (HCDR2) comprises or consists of an amino acid sequence of SEQ ID NO: 78; optionally with one, two or three modification(s) selected from substitution(s), addition(s), deletion(s) and any combination thereof;
the heavy chain CDR3 (HCDR3) comprises or consists of an amino acid sequence of SEQ ID NO: 79; optionally with one, two or three modification(s) selected from substitution(s), addition(s), deletion(s) and any combination thereof;
the light chain CDR1 (LCDR1) comprises or consists of an amino acid sequence of SEQ ID NO: 81; optionally with one, two or three modification(s) selected from substitution(s), addition(s), deletion(s) and any combination thereof;
the light chain CDR2 (LCDR2) comprises or consists of an amino acid sequence of SEQ ID NO: 82; optionally with one, two or three modification(s) selected from substitution(s), addition(s), deletion(s) and any combination thereof; and
the light chain CDR3 (LCDR3) comprises or consists of an amino acid sequence of SEQ ID NO: 82, optionally with one, two or three modification(s) selected from substitution(s), addition(s), deletion(s) and any combination thereof.

In another aspect, the antigen-binding domain comprises or consists essentially of:
(i) a heavy chain comprising a CDR1 of SEQ ID NO: 77, a CDR2 of SEQ ID NO: 78 and a CDR3 of SEQ ID NO: 79; and (ii) a light chain comprising a CDR1 of SEQ ID NO: 81, a CDR2 of SEQ ID NO: 82 and a CDR3 of SEQ ID NO: 83.

In an aspect, the antigen-binding domain comprises or consists essentially of:
(a) a heavy chain variable region (VH) comprising or consisting of an amino acid sequence of SEQ ID NO: 76, optionally with one, two or three modification(s) selected from substitution(s), addition(s), deletion(s) and any combination thereof;
(b) a light chain variable region (VL) comprising or consisting of an amino acid sequence of SEQ ID NO: 80, optionally with one, two or three modification(s) selected from substitution(s), addition(s), deletion(s) and any combination thereof.

Particular combinations of targets specifically expressed on immune cells surface and of immune-stimulating moieties are contemplated herein.

For instance, the bifunctional molecule comprises an immune-stimulating moiety being SIRPα or SIRPγ, a fragment or a variant thereof and the antigen binding domain does not bind PD-L1 or PD-L2. In particular, the bifunctional molecule comprises an immune-stimulating moiety being SIRPα or SIRPγ, a fragment or a variant thereof and an antigen binding domain that binds (and preferably antagonizes) PD-1, CTLA-4 or TIM3, especially PD-1. Optionally, the bifunctional molecule comprises one of the following combinations: a) an antigen binding domain that binds PD-1 and SIRPα or a fragment or variant thereof; b) an antigen binding domain that binds CTLA-4 and SIRPα or a fragment or variant thereof; c) an antigen binding domain that binds TIM3 and SIRPα or a fragment or variant thereof; d) an antigen binding domain that binds PD-1 and SIRPγ or a fragment or variant thereof; e) an antigen binding domain that binds CTLA-4 and SIRPγ or a fragment or variant thereof; or f) an antigen binding domain that binds TIM3 and SIRPγ or a fragment or variant thereof.

### Peptide Linker

In a particular aspect, the bifunctional molecule according to the invention further comprises a peptide linker connecting the antigen binding domain and the immune-stimulating moiety to the Fc chain. The peptide linker usually has a length and flexibility enough to ensure that the immune-stimulating moiety and the antigen binding domain connected with the linker in between have enough freedom in space to exert their functions.

In an aspect of the disclosure, the immune-stimulating moiety is preferably linked to the Fc chain through a peptide linker. In an aspect of the disclosure, the antigen binding domain can be linked to the Fc chain by the hinge naturally found in a heavy chain for connecting VH domain, especially CH1 domain to the CH2 domain of the Fc chain.

As used herein, the term “linker” refers to a sequence of at least one amino acid. Such a linker may be useful to prevent steric hindrances. The linker is usually 3-44 amino acid residues in length. Preferably, the linker has 3-30 amino acid residues. In some aspects, the linker has 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues.

The linker sequence may be a naturally occurring sequence or a non-naturally occurring sequence. If used for therapeutic purposes, the linker is preferably non-immunogenic in the subject to which the bifunctional molecule is administered. One useful group of linker sequences are linkers derived from the hinge region of heavy chain antibodies as described in WO 96/34103 and WO 94/04678. Other examples are poly-alanine linker sequences. Further preferred examples of linker sequences are Gly/Ser linkers of different length including (Gly4Ser)4, (Gly4Ser)3, (Gly4Ser)2, Gly4Ser, Gly3Ser, Gly3, Gly2ser and (Gly3Ser2)3, in particular (Gly4Ser)3. Preferably, the linker is selected from the group consisting of (Gly4Ser)4, (Gly4Ser)3, and (Gly3Ser2)3. Even more preferably, the linker is (GGGGS)3.

In one aspect, the linker comprised in the bifunctional molecule is selected in the group consisting of (Gly4Ser)4, (Gly4Ser)3, (Gly4Ser)2, Gly4Ser, Gly3Ser, Gly3, Gly2ser and (Gly3Ser2)3, preferably is (Gly4Ser)3. Preferably, the linker is selected from the group consisting of (Gly4Ser)4, (Gly4Ser)3, and (Gly3Ser2)3.

### Fc Domain

The Fc domain of the bifunctional molecule can be part of the antigen binding domain, especially a heavy chain of an IgG immunoglobulin. Indeed, when the antigen binding domain is a Fab, the bifunctional molecule may comprise one heavy chain, including the variable heavy chain (VH), CH1, hinge, CH2 and CH3 domains. However, the bifunctional molecule may also have other structures such as scFv, or diabody. For instance, it may comprise an Fc domain linked to antibody derivative such as.

The Fc domain can be derived from a heavy chain constant domain of a human immunoglobulin heavy chain, for example, IgG1, IgG2, IgG3, IgG4, or other classes. Preferably, the bifunctional molecule comprises an IgG1 or an IgG4 heavy chain constant domain.

Preferably, the Fc domain comprises CH2 and CH3 domains. Optionally, it can include all or a portion of the hinge region, the CH2 domain and/or the CH3 domain. In some aspects, the CH2 and/or a CH3 domains are derived from a human IgG4 or IgG1 heavy chain. Preferably, the Fc domain includes all or a portion of a hinge region. The hinge region can be derived from an immunoglobulin heavy chain, e.g., IgG1, IgG2, IgG3, IgG4, or other classes. Preferably, the hinge region is derived from human IgG1, IgG2, IgG3, IgG4. More preferably, the hinge region is derived from a human or humanized IgG1 or IgG4 heavy chain.

The IgG1 hinge region has three cysteines, two of which are involved in disulfide bonds between the two heavy chains of the immunoglobulin. These same cysteines permit efficient and consistent disulfide bonding formation between Fc portions. Therefore, a preferred hinge region of the present invention is derived from IgG1, more preferably from human IgG1. In some aspects, the first cysteine within the human IgG1 hinge region is mutated to another amino acid, preferably serine.

The hinge region of IgG4 is known to form interchain disulfide bonds inefficiently. However, a suitable hinge region for the present invention can be derived from the IgG4 hinge region, preferably containing a mutation that enhances correct formation of disulfide bonds between heavy chain-derived moieties (Angal S, et al. (1993) Mol. Immunol., 30:105-8). More preferably, the hinge region is derived from a human IgG4 heavy chain.

The bifunctional molecule comprises a dimeric Fc domain. Accordingly, two monomers comprise each one a Fc chain, the Fc chains being able to form a dimeric Fc domain. The dimeric Fc domain can be an homodimer, each Fc monomer being identical or essentially identical. Alternatively, the dimeric Fc domain can be a heterodimer, each Fc monomer being different and complementary in order to promote the formation of the heterodimeric Fc domain.

More specifically, the Fc domain is a heterodimeric Fc domain. Heterodimeric Fc domains are made by altering the amino acid sequence of each monomer. The heterodimeric Fc domains rely on amino acid variants in the constant regions that are different on each chain to promote heterodimeric formation and/or allow for ease of purification of heterodimers over the homodimers. There are a number of mechanisms that can be used to generate the heterodimers of the present invention. In addition, as will be appreciated by those in the art, these mechanisms can be combined to ensure high heterodimerization. Thus, amino acid variants that lead to the production of heterodimers are referred to as “heterodimerization variants”. Heterodimerization variants can include steric variants (e.g. the “knobs and holes” or “skew” variants described below and the “charge pairs” variants described below) as well as “pi variants”, which allows purification of homodimers away from heterodimers.

WO2014/145806, hereby incorporated by reference in its entirety, discloses useful mechanisms for heterodimerization include “knobs and holes”, “electrostatic steering” or “charge pairs”, pi variants, and general additional Fc variants. See also, Ridgway et al., Protein Engineering 9(7):617 (1996); Atwell et al., J. Mol. Biol. 1997 270:26; U.S. Pat. No. 8,216,805, Merchant et al., Nature Biotech. 16:677 (1998), all of which are hereby incorporated by reference in their entirety. For “electrostatic steering” see Gunasekaran et al., J. Biol. Chem. 285(25): 19637 (2010), hereby incorporated by reference in its entirety. For pi variants, see US 2012/0149876 hereby incorporated by reference in its entirety.

Then, in a preferred aspect, the heterodimeric Fc domain comprises a first Fc chain and a complementary second Fc chain based on the “knobs and holes” technology. For instance, the first Fc chain is a “knob” or K chain, meaning that it comprises the substitution characterizing a knob chain, and the second Fc chain is a “hole” or H chain, meaning that it comprises the substitution characterizing a hole chain. And vice versa, the first Fc chain is a “hole” or H chain, meaning that it comprises the substitution characterizing a hole chain, and the second Fc chain is a “knob” or K chain, meaning that it comprises the substitution characterizing a knob chain. In a preferred aspect, the first Fc chain is a “hole” or H chain and the second Fc chain is a “knob” or K chain.

Optionally, the heterodimeric Fc domain may comprise one heterodimeric Fc chain which comprises the substitutions as shown in the following table F and the other heterodimeric Fc chain comprising the substitutions as shown in the following table F.

TABLE F
(the numbering being according to EU index)

| | The complementary Fc |
|---|---|
| Fc chain having the | chain having the |
| following substitutions | following substitutions |
| (Hole chain or H chain) | (Knob chain or K chain) |
| D221E/P228E/L368E | D221R/P228R/K409R |
| C220E/P228E/368E | C220R/E224R/P228R/K409R |
| S364K/E357Q | L368D/K370S |
| L368D/K370S | S364K |
| L368E/K370S | S364K |
| T411T/E360E/Q362E | D401K |
| L368D/K370S | S364K/E357L |
| K370S | S364K/E357Q |
| T366S/L368A/Y407V | T366W |
| T366S/L368A/Y407V/Y349C | T366W/S354C |
| F368D/K370S | S364K |
| F368D/K370S | S364K/E357F |
| F368D/K370S | S364K/E357Q |
| T411E/K360E/Q362E | D401K |
| F368E/K370S | S364K |
| K370S | S364K/E357Q |
| T366S/F368A/Y407V | T366W |
| T366S/L368A/Y407V/Y349C | T366W/S354C |

In a preferred aspect, the first Fc chain is a “hole” or H chain and comprises the substitutions T366S/L368A/Y407V/Y349C and the second Fc chain is a “knob” or K chain and comprises the substitutions T366W/S354C.

Optionally, the Fc chain may further comprise additional substitutions.

In particular, for bifunctional molecules that target cell-surface molecules, especially those on immune cells, abrogating effector functions may be required. Engineering Fc regions may also be desired to either reduce or increase the effector function of the bifunctional molecules.

In certain aspects, amino acid modifications may be introduced into the Fc region to generate an Fc region variant. In certain aspects, the Fc region variant possesses some, but not all, effector functions. Such bifunctional molecules may be useful, for example, in applications in which the half-life of the antibody in vivo is important, yet certain effector functions are unnecessary or deleterious. Numerous substitutions or substitutions or deletions with altered effector function are known in the art.

In one aspect, the constant region of the Fc domain contains a mutation that reduces affinity for an Fc receptor or reduces Fc effector function. For example, the constant region can contain a mutation that eliminates the glycosylation site within the constant region of an lgG heavy chain. Preferably, the CH2 domain contains a mutation that eliminates the glycosylation site within the CH2 domain.

In a particular aspect, the Fc domain is modified to increase the binding to FcRn, thereby increasing the half-life of the bifunctional molecule. In another aspect or additional aspect, the Fc domain is modified to decrease the binding to FcyR, thereby reducing ADCC or CDC, or to increase the binding to FcyR, thereby increasing ADCC or CDC.

The alteration of amino acids near the junction of the Fc portion and the non-Fc portion can dramatically increase the serum half-life of the Fc fusion protein as shown in WO 01/58957. Accordingly, the junction region of a protein or polypeptide of the present invention can contain alterations that, relative to the naturally-occurring sequences of an immunoglobulin heavy chain and erythropoietin, preferably lie within about 10 amino acids of the junction point. These amino acid changes can cause an increase in hydrophobicity. In one embodiment, the constant region is derived from an lgG sequence in which the C-terminal lysine residue is replaced. Preferably, the C-terminal lysine of an IgG sequence is replaced with a non-lysine amino acid, such as alanine or leucine, to further increase serum half-life.

In one embodiment, the constant region of the Fc domain has one of the mutations described in the Table

G below, or any combination thereof.

TABLE G
Suitable human engineered Fc domain of an antibody. numbering of residues in the heavy chain
constant region is according to EU numbering (Edelman, G. M. et al., Proc. Natl. Acad. USA, 63,
78-85 (1969); www.imgt.org/IMGTScientificChart/Numbering/Hu_IGHGnber.html#refs)

| Engineered | | | | |
|---|---|---|---|---|
| Fc | Isotype | Mutations | FcR/C1q Binding | Effector Function |
| hIgG1e1-Fc | IgG1 | T250Q/M428L | Increased | Increased half-life |
| | | | binding to FcRn | |
| hIgG1e2-Fc | IgG1 | M252Y/S254T/T256E + | Increased | Increased half-life |
| | | H433K/N434F | binding to FcRn | |
| hIgG1e3-Fc | IgG1 | E233P/L234V/L235A/G236A + | Reduced binding | Reduced ADCC and |
| | | A327G/A330S/P331S | to FcγRI | CDC |
| hIgG1e4-Fc | IgG1 | E333A | Increased | Increased ADCC and |
| | | | binding to | CDC |
| | | | FcγRIIIa | |
| hIgG1e5-Fc | IgG1 | S239D/A330L/1332E | Increased | Increased ADCC |
| | | | binding to | |
| | | | FcγRIIIa | |
| hIgG1e6-Fc | IgG1 | P2571/Q311 | Increased | Unchanged half-life |
| | | | binding to FcRn | |
| hIgG1e7-Fc | IgG1 | K326W/E333S | Increased | Increased CDC |
| | | | binding to C1q | |
| hIgG1e9-Fc | IgG1 | S239D/1332E/G236A | Increased | Increased |
| | | | FcγRIIa/FcγRIIb | macrophage |
| | | | ratio | phagocytosis |
| hIgG1e9-Fc | IgG1 | N297A | Reduced binding | Reduced ADCC and |
| | | | to FcγRI | CDC |
| hIgG1e9-Fc | IgG1 | LALA (L234A/L235A) | Reduced binding | Reduced ADCC and |
| | | | to FcγRI | CDC |
| hIgG1e10-Fc | IgG1 | N297A + YTE | Reduced binding | Reduced ADCC and |
| | | (N298A + | to FcγRI | CDC |
| | | M252Y/S254T/T256E) | Increased | Increased half-life |
| | | | binding to FcRn | |
| hIgG1e11-Fc | IgG1 | K322A | Reduced binding | Reduced CDC |
| | | | to C1q | |
| hIgG1e12-Fc | IgG1 | N297A + YTE | | Reduced ADCC and |
| | | (N298A + | | CDC |
| | | M252Y/S254T/T256E) + | | Increased half-life |
| | | K444A | | Abolish cleveage of |
| | | | | the C-terminal lysine |
| | | | | of the antbody |
| hIgG4e1-Fc | IgG4 | S228P | — | Reduced Fab-arm |
| | | | | exchange |
| hIgG4e1-Fc | IgG4 | LALA (L234A/L235A) | Increased | Increased half-life |
| | | | binding to FcRn | |
| hIgG4e2-Fc | IgG4 | S228P+ YTE (S228P + | — | Reduced Fab-arm |
| | | M252Y/S254T/T256E) | Increased | exchange |
| | | | binding to FcRn | Increased half-life |
| hIgG4e3-Fc | IgG4 | N297A + YTE | | Reduced ADCC and |
| | | (N298A + | | CDC |
| | | M252Y/S254T/T256E) + | | Increased half-life |
| | | K444A | | Abolish cleveage of |
| | | | | the C-terminal lysine |
| | | | | of the antibody |

In a particular aspect, the bifunctional molecule comprises a human IgG1 heavy chain constant domain or an IgG1 Fc domain, optionally with a substitution or a combination of substitutions selected from the group consisting of T250Q/M428L; M252Y/S254T/T256E+H433K/N434F; E233P/L234V/L235A/G236A+A327G/A330S/P331S; E333A; S239D/A330L/1332E; P2571/Q311; K326W/E333S; S239D/1332E/G236A; N297A; L234A/L235A; P329G; N297A+M252Y/S254T/T256E; K322A and K444A, preferably selected from the group consisting of N297A optionally in combination with M252Y/S254T/T256E, and L234A/L235A optionally with P329G.

In a particular aspect, the bifunctional molecule comprises a human IgG1 heavy chain constant domain or an IgG1 Fc domain, optionally with a substitution or a combination of substitutions selected from the group consisting of T250Q/M428L; M252Y/S254T/T256E+H433K/N434F; E233P/L234V/L235A/G236A +A327G/A330S/P331S; E333A; S239D/A330L/1332E; P2571/Q311; K326W/E333S; S239D/1332E/G236A; N297A; L234A/L235A; P329G; N297A+M252Y/S254T/T256E; K322A,K444A, K444E, K444D, K444G, K444S, M428L, L309D, Q311H, N434S, M428L+N434S and L309D+Q311H+N434S, preferably selected from the group consisting of N297A optionally in combination with M252Y/S254T/T256E, and L234A/L235A optionally with P329G.

The bifunctional molecule comprising a human IgG1 heavy chain constant domain or an IgG1 Fc domain with the combination of substitutions L234A/L235A/P329G greatly reduces or altogether suppresses ADCC, ADCP and/or CDC caused by said bifunctional molecule, thus reducing nonspecific cytotoxicity.

In another aspect, the bifunctional molecule comprises a human IgG4 heavy chain constant domain or a human IgG4 Fc domain, optionally with a substitution or a combination of substitutions selected from the group consisting of S228P; L234A/L235A, P329G, S228P+M252Y/S254T/T256E and K444A. Even more preferably, the bifunctional molecule, comprises an IgG4 Fc-region with a S228P that stabilizes the IgG4.

In another aspect, the bifunctional molecule comprises a human IgG4 heavy chain constant domain or a human IgG4 Fc domain, optionally with a substitution or a combination of substitutions selected from the group consisting of S228P; L234A/L235A; L234A/L235A/P329G, P329G, S228P+M252Y/S254T/T256E, K444A K444E, K444D, K444G and K444S. Even more preferably, the bifunctional molecule, preferably the bifunctional molecule according to the invention comprises an IgG4 Fc-region with a S228P that stabilizes the IgG4.

As mentioned herein the “/” and “+” refer to mutations that are cumulative. Thus, by the mutation S228P+M252Y/S254T/T256E, it is meant the following mutations: S228P, M252Y, S254T and T256E.

The bifunctional molecule comprising a human IgG4 heavy chain constant domain or an IgG4 Fc domain with the substitution P329G reduces ADCC and/or CDC caused by said bifunctional molecule, thus reducing nonspecific cytotoxicity.

All subclass of Human IgG carries a C-terminal lysine residue of the antibody heavy chain (K444) that are susceptible to be cleaved off in circulation. This cleavage in the blood may compromise or decrease the bioactivity of the bifunctional molecule by releasing the linked immune-stimulating moiety to the bifunctional molecule. To circumvent this issue, K444 amino acid in the lgG domain can be substituted by another amino acid to reduce proteolytic cleavage, a mutation commonly used for antibodies. Then, in one aspect, the bifunctional molecule comprises at least one further amino acid substitution consisting of K444A, K444E, K444D, K444G or K444S, preferentially K444A. Particularly, K444 amino acid in the lgG domain can be substituted by an alanine to reduce proteolytic cleavage, a mutation commonly used for antibodies. Then, in one aspect, the bifunctional molecule comprises at least one further amino acid substitution consisting of K444A.

Optionally, the bifunctional molecule comprises an additional cysteine residue at the C-terminal domain of the Fc domain to create an additional disulfide bond and potentially restrict the flexibility of the bifunctional molecule.

In one aspect, the bifunctional molecule comprises one heavy chain constant domain of SEQ ID NO: 39 or 52 and/or one light chain constant domain of SEQ ID NO: 40, particularly one heavy chain constant domain or Fc domain of SEQ ID NO: 39 or 52 and one light chain constant domain of SEQ ID NO: 40, particularly such as disclosed in Table I below.

TABLE H

| | |
|---|---|
| Heavy chain | ASTKGPSVFPLAPCSRSTSESTAALGCLVKD |
| constant domain | YFPEPVTVSWNSGALTSGVHTFPAVLQSSGL |
| (IgG4m-S228P) | YSLSSVVTVPSSSLGTKTYTCNVDHKPSNTK |
| SEQ ID NO: 39 | VDKRVESKYGPPCPPCPAPEFLGGPSVFLFP |
| | PKPKDTLMISRTPEVTCVVVDVSQEDPEVQF |
| | NWYVDGVEVHNAKTKPREEQFNSTYRVVSVL |
| | TVLHQDWLNGKEYKCKVSNKGLPSSIEKTIS |
| | KAKGQPREPQVYTLPPSQEEMTKNQVSLTCL |
| | VKGFYPSDIAVEWESNGQPENNYKTTPPVLD |
| | SDGSFFLYSRLTVDKSRWQEGNVFSCSVMHE |
| | ALHNHYTQKSLSLSPGK |
| Light chain | RTVAAPSVFIFPPSDEQLKSGTASVVCLLNN |
| constant domain | FYPREAKVQWKVDNALQSGNSQESVTEQDSK |
| (CLkappa) | DSTYSLSSTLTLSKADYEKHKVYACEVTHQG |
| SEQ ID NO: 40 | LSSPVTKSFNRGEC |
| Heavy chain | ASTKGPSVFPLAPSSKSTSGGTAALGCLVKD |
| constant domain | YFPEPVTVSWNSGALTSGVHTFPAVLQSSGL |
| (IgG1m-N298A) | YSLSSVVTVPSSSLGTQTYICNVNHKPSNTK |
| SEQ ID NO: 52 | VDKKVEPKSCDKTHTCPPCPAPELLGGPSVF |
| | LFPPKPKDTLMISRTPEVTCVVVDVSHEDPE |
| | VKFNWYVDGVEVHNAKTKPREEQYASTYRVV |
| | SVLTVLHQDWLNGKEYKCKVSNKALPAPIEK |
| | TISKAKGQPREPQVYTLPPSRDELTKNQVSL |
| | TCLVKGFYPSDIAVEWESNGQPENNYKTTPP |
| | VLDSDGSFFLYSKLTVDKSRWQQGNVFSCSV |
| | MHEALHNHYTQKSLSLSPGK |

Example of a heavy chain constant domain and a light chain constant domain suitable for the bifunctional molecules according to the invention.

In one particular aspect, the bifunctional molecule according to the invention comprises a heterodimer of Fc domains that comprises the “knob into holes” modifications such as described above. Preferably, such Fc domains are IgG1 or IgG4 Fc domain such as described above, even more preferably an IgG1 Fc domain comprising the mutation N297A such as disclosed above.

For instance, the first Fc chain is a “hole” or H chain and comprises the substitutions T366S/L368A/Y407V/Y349C and optionally N297A and the second Fc chain is a “knob” or K chain and comprises the substitutions T366W/S354C and optionally N297A. Preferably, the first Fc chain is a “hole” or H chain and comprises the substitutions T366S/L368A/Y407V/Y349C and N297A and the second Fc chain is a “knob” or K chain and comprises the substitutions T366W/S354C and N297A. More particularly, the second Fc chain may comprise or consist in SEQ ID NO: 14 and/or the first Fc chain may comprise or consist in SEQ ID NO: 13.

Particularly, the first Fc chain is a “hole” or H chain and comprises the substitutions T366S/L368A/Y407V/Y349C and N297A and the second Fc chain is a “knob” or K chain and comprises the substitutions T366W/S354C and N297A. More particularly, the second Fc chain may comprise or consist in SEQ ID NO: 14 and/or the first Fc chain may comprise or consist in SEQ ID NO: 13.

More specifically, the immune-stimulating moiety according to the invention is linked to the knob-chain and/or the hole chain of the heterodimeric Fc domain. Thus, the bifunctional molecule according to the invention may comprises a single immune-stimulating moiety either linked to the hole-chain or to the knob-chain of the Fc domain. Preferably, the bifunctional molecule according to the invention comprises a single immune-stimulating moiety linked to the hole-chain of the Fc domain.

In a first aspect, the bifunctional molecule comprises an immune-stimulating moiety linked to the C-terminal of the knob-chain of the Fc domain, such knob-chain of the Fc domain being linked to an antigen binding domain.

In a second aspect, the bifunctional molecule comprises an immune-stimulating moiety linked to the C-terminal of the hole-chain of the Fc domain, such hole-chain of the Fc-domain being linked to an antigen binding domain at its N-terminal end.

Optionally, the bifunctional molecule comprises a single immune-stimulating moiety linked to the C-terminal of the hole-chain of the Fc domain, wherein the bifunctional molecule comprises only a single antigen binding domain linked in the N-terminal end of the hole chain of the Fc domain. In such aspect, the knob chain domain is devoid of immune-stimulating moiety and of an antigen binding domain.

Optionally, the bifunctional molecule comprises a single immune-stimulating moiety linked to the C-terminal of the knob-chain of the Fc domain, wherein the bifunctional molecule comprises only a single antigen binding domain linked in the N-terminal end of the knob chain of the Fc domain. In such aspect, the hole chain domain is devoid of immune-stimulating moiety and of an antigen binding domain.

Accordingly, an object of the present invention relates to a polypeptide comprising from the N-terminal to the C-terminal an antigen binding domain (or at least the part therefor corresponding to the heavy chain), a Fc chain (knob or hole Fc chain), preferably the hole-chain of the Fc domain, and an immune-stimulating moiety. The complementary chain comprises a complementary Fc chain devoid of immune-stimulating moiety and of antigen binding domain, preferably the knob-chain of the Fc domain.

In a very particular aspect, the bifunctional molecule targets PD-1 and comprises:
(a) a heavy chain comprising or consisting of an amino acid sequence selected from the group consisting of SEQ ID NO: 29, 30, 31, 32, 33, 34, 35 or 36, optionally with one, two or three modification(s) selected from substitution(s), addition(s), deletion(s) and any combination thereof at any position but positions 7, 16, 17, 20, 33, 38, 43, 46, 62, 63, 65, 69, 73, 76, 78, 80, 84, 85, 88, 93, 95, 96, 97, 98, 100, 101, 105, 106 and 112 of SEQ ID NO: 29, 30, 31, 32, 33, 34, 35 or 36, respectively, and the substitutions corresponding to the hole or knob chain, preferably the hole chain, more specifically as disclosed in Table F, in particular, in SEQ ID NO: 29, 30, 31, 32, 33, 34, 35 or 36, either T363S/L365A/Y4047V/Y346C or T363W/S351C, preferably T363S/L365A/Y4047V/Y346C, and optionally N294A in any of SEQ ID NO: 29, 30, 31, 32, 33, 34, 35 or 36;
(b) a light chain comprising or consisting of an amino acid sequence of SEQ ID NO: 37 or SEQ ID NO: 38, optionally with one, two or three modification(s) selected from substitution(s), addition(s), deletion(s) and any combination thereof at any position but positions 3, 4, 7, 14, 17, 18, 28, 29, 33, 34, 39, 42, 44, 50, 81, 88, 94, 97, 99 and 105 of SEQ ID NO: 37 or SEQ ID NO: 38.

In another aspect, the bifunctional molecule comprises or consists in any of the following combinations of a heavy chain (CH) and a light chain (CL):

| CH (SEQ ID NO:), optionally with one, two or | |
|---|---|
| three modification(s) selected from | |
| substitution(s), addition(s), deletion(s) at any | CL (SEQ ID NO:), optionally with one, two or |
| position but positions 7, 16, 17, 20, 33, 38, 43, | three modification(s) selected from |
| 46, 62, 63, 65, 69, 73, 76, 78, 80, 84, 85, 88, 93, | substitution(s), addition(s), deletion(s) at any |
| 95, 96, 97, 98, 100, 101, 105, 106 and 112 of | position but positions 3, 4, 7, 14, 17, 18, 28, 29, |
| SEQ ID NO: 29, 30, 31, 32, 33, 34, 35 or 36 of | 33, 34, 39, 42, 44, 50, 81, 88, 94, 97, 99 and |
| SEQ ID NO: | 105of SEQ ID NO: |
| 27 | 37 |
| 27 | 38 |
| 28 | 37 |
| 28 | 38 |
| 29 | 37 |
| 29 | 38 |
| 30 | 37 |
| 30 | 38 |
| 31 | 37 |
| 31 | 38 |
| 32 | 37 |
| 32 | 38 |
| 33 | 37 |
| 33 | 38 |
| 34 | 37 |
| 34 | 38 |
| 35 | 37 |
| 35 | 38 |
| 36 | 37 |
| 36 | 38 |

with the heavy chain comprising the substitutions corresponding to the hole or knob chain, preferably the hole chain, more specifically as disclosed in Table F, in particular, in SEQ ID NO: 29, 30, 31, 32, 33, 34, 35 or 36, in particular either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering.

In a very particular aspect, the bifunctional molecule targets PD-1 and comprises a light chain comprising or consisting of SEQ ID NO: 37 or 38.

Accordingly, the bifunctional molecule may comprise one heavy chain comprising any of the SEQ ID NOs: 29, 30, 31, 32, 33, 34, 35 and 36, the Fc chain being optionally modified to promote a heterodimerization of the Fc chains for forming a heterodimeric Fc domain. More specifically, the heavy chain comprises the substitutions corresponding to the hole or knob chain, preferably the hole chain, more specifically as disclosed in Table F, particularly either T366S/L368A/Y407V/Y349C or T366W/S354C, preferably T366S/L368A/Y407V/Y349C, and optionally N297A in any of SEQ ID NO: 29, 30, 31, 32, 33, 34, 35 or 36, the positions of the substitutions being defined according to EU numbering. The heavy chain is linked, optionally via a linker, at its C terminal end to the immune-stimulating moiety.

In a very particular aspect, the bifunctional molecule comprises a light chain comprising or consisting of SEQ ID NO: 38 and one heavy chain comprising SEQ ID NO: 35, the Fc chain being optionally modified to promote a heterodimerization of the Fc chains for forming a heterodimeric Fc domain. In one aspect, the heavy chain is linked, optionally via a linker, at its C terminal end to the immune-stimulating moiety. In an alternative aspect, the light chain is linked, optionally via a linker, at its C terminal end to the immune-stimulating moiety.

### Examples of Particular Constructions

The molecule according to the invention may comprise any of the immune-stimulating moiety disclosed herein, preferably any SIRP molecule (i.e. SIRPγ, SIRPα or SIRPβ) disclosed herein.

In addition, the molecule according to the invention may comprise any of the antigen binding domain disclosed herein, preferably any single anti-PD1 or anti-CD28 antibody, in particular derived from any anti-PD-1 or anti-CD28 antibody disclosed herein.

Such molecule comprising a SIRP molecule and a single anti-PD-1 or anti-CD28 antigen binding domain may further comprises any particular peptide linker, hinge and/or Fc domain disclosed herein.

Preferably, the molecule comprises a single anti PD-1 or anti-CD28 antigen binding domain and a single SIRP molecule. For example, this construction is also called anti PD-1*1 SIRP*1 or anti-CD28*1 SIRP*1, respectively.

In a particular, the molecule according to the invention comprises a first monomer comprising an antigen-binding domain covalently linked to a first Fc chain optionally via a peptide linker, said first Fc chain being covalently linked to a SIRP molecule (i.e. SIRPγ, SIRPα or SIRPβ), optionally via a peptide linker, and a second monomer comprising a second Fc chain complementary to the first Fc chain, and preferably devoid of antigen-binding domain and of a SIRP molecule, said first and second Fc chains forming a dimeric Fc domain.

More particularly, the molecule comprises a first monomer comprising an antigen-binding domain covalently linked via C-terminal end to N-terminal end of a first heterodimeric Fc chain optionally via a peptide linker, said first heterodimeric Fc chain being covalently linked by its C-terminal end to the N-terminal end a SIRP molecule (i.e. SIRPγ, SIRPα or SIRPβ), optionally via a peptide linker, and a second monomer comprising a second heterodimeric Fc chain complementary to the first heterodimeric Fc chain and devoid of antigen-binding domain and of a SIRP molecule (i.e. SIRPγ, SIRPα or SIRPβ), preferably devoid of any other molecule, in particular of any other immune-stimulating moiety. Such a molecule is for example illustrated under the name “Construct 2” in FIG. 1.

Alternatively, the molecule comprises a first monomer comprising an antigen-binding domain covalently linked via C-terminal end to N-terminal end of a first heterodimeric Fc chain optionally via a peptide linker, light chain of said antigen-binding domain being covalently linked by its C-terminal end to the N-terminal end a SIRP molecule (i.e. SIRPγ, SIRPα or SIRPβ), optionally via a peptide linker, and a second monomer comprising a second heterodimeric Fc chain complementary to the first heterodimeric Fc chain and devoid of antigen-binding domain and of a SIRP molecule (i.e. SIRPγ, SIRPα or SIRPβ), preferably devoid of any other molecule, in particular of any other immune-stimulating moiety.

Particularly, the antigen-binding domain comprises a light chain covalently linked by its C-terminal end to the N-terminal end a SIRP molecule (i.e. SIRPγ, SIRPα or SIRPβ), optionally via a peptide linker; and a heavy chain moiety of the antigen-binding domain (i.e., VH+CH1) covalently linked via C-terminal end to N-terminal end of a first heterodimeric Fc chain. Such a molecule is for example illustrated under the name “Construct 3” in FIG. 1.

Particularly, the single anti PD-1 antigen binding domain derives from an anti-PD-1 antibody selected from the group consisting of Pembrolizumab, Nivolumab, Pidilizumab, Cemiplimab, Camrelizumab, AUNP12, AMP-224, BGB-A317, spartalizumab, MK-3477, PF-06801591, JNJ-63723283, genolimzumab, LZM-009, BCD-100, SHR-1201, BAT-1306, AK-103, MEDI-0680, MEDI0608, JS001, BI-754091, TSR-042, GLS-010, AM-0001, STI-1110, AGEN2034, MGA012, or IBI308, 5C4, 17D8, 2D3, 4H1, 4A11, 7D3, and 5F4.

Preferably, the single anti PD-1 antigen binding domain comprises or consists essentially of: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or 65, a CDR2 of SEQ ID NO: 66 and a CDR3 of SEQ ID NO: 16.

More preferably, the antigen binding domain comprises or consists essentially of: (i) a heavy chain comprising a CDR1 of SEQ ID NO: 51, a CDR2 of SEQ ID NO: 53 and a CDR3 of SEQ ID NO: 61; and (ii) a light chain comprising a CDR1 of SEQ ID NO: 65, a CDR2 of SEQ ID NO: 66 and a CDR3 of SEQ ID NO: 16.

Preferentially, the antigen-binding domain comprises or consists essentially of: (a) a heavy chain variable region (VH) comprising or consisting of an amino acid sequence of SEQ ID NO: 18, 19, 20, 21, 22, 23, 24 or 25, preferably SEQ ID NO: 24; and (b) a light chain variable region (VL) comprising or consisting of an amino acid sequence of SEQ ID NO: 27 or SEQ ID NO: 28, preferably SEQ ID NO: 28.

In particular, the single anti PD-1 antigen binding domain comprises or consists essentially of a variable heavy chain (VH) as defined in SEQ ID NO: 24 and a variable light chain (VL) as defined in SEQ ID NO: 28.

In addition to the single anti-PD-1 domain, the bifunctional molecule according to the invention comprises a immune-stimulating moiety that is a SIRP molecule, preferably selected from the group consisting of SIRPγ, SIRPα or SIRPβ or any fragments and variants thereof.

Particularly, the single anti-CD28 antigen binding domain derives from an anti-CD28 antibody selected from the group consisting of Theralizumab (TGN1412), TGN1112, CD28.1 to CD28.6 as disclosed in Nunes et al. International immunology vol. 5,3 (1993): 311-5, Anc28.1 as disclosed in Waibler et al.PLOS ONE (2008) 3(3): e1708, and anti-CD28 as disclosed in EP3941941, EP3810282, EP3898695, EP3897715 or WO2022/061098.

Preferably, the single anti-CD28 antigen binding domain comprises or consists essentially of: (i) a heavy chain comprising a CDR1 of SEQ ID NO: 77, a CDR2 of SEQ ID NO: 78 and a CDR3 of SEQ ID NO: 79; and (ii) a light chain comprising a CDR1 of SEQ ID NO: 81, a CDR2 of SEQ ID NO: 82 and a CDR3 of SEQ ID NO: 83. Preferentially, the antigen-binding domain comprises or consists essentially of: (a) a heavy chain variable region (VH) comprising or consisting of an amino acid sequence of SEQ ID NO: 76; and (b) a light chain variable region (VL) comprising or consisting of an amino acid sequence of SEQ ID NO: 80.

In addition to the single anti-CD28 antigen binding domain, the molecule comprises a immune-stimulating moiety that is a SIRP molecule, preferably selected from the group consisting of SIRPγ, SIRPα or SIRPβ or any fragments and variants thereof.

The SIRPα or SIRPγ variant may comprise i) D1 domain of SIRPγ, D2 and D3 domains of SIRPα ii) D1 and D2 domains of SIRPγ and D3 domain of SIRPα iii) D1 domain of SIRPγ, D2 domain of SIRPα and D3 domain of SIRPγ, iv) D1 domain of SIRPα, D2 and D3 domains of SIRPγ, v) D1 and D2 domains of SIRPα and D3 domain of SIRPg or vi) D1 domain of SIRPα, D2 domain of SIRPγ and D3 domain of SIRPα. The SIRPα or SIRPγ fragment of the extracellular domain preferably comprises or consists of binding regions or of the amino acids within the set of contact residues that interact with CD47.

In an embodiment, the SIRP molecule is a human SIRPα or fragments and variants thereof. For instance, the SIRPα fragment may consist of its extracellular domain, even more preferably consists essentially of the 31 to 373 amino acids of the wild-type human SIRPα, in particular SEQ ID NO: 1. Preferably, the extracellular part of SIRPα has a sequence as shown in SEQID NO: 1 or a sequence having at least 80, 85, 90, 91, 92, 93, 94, 95, 96, 97, 98 or 99% of identity with SEQ ID NO: 1 or having 1 to 10 modifications selected from the group consisting of addition, deletion, substitution and combinations thereof with respect to the sequence of SEQ ID NO: 1.

Alternatively, the immune stimulating moiety is SIRPγ, a fragment thereof, especially the extracellular part thereof, or a variant of SIRPγ or a fragment thereof. Preferably, the extracellular part of SIRPγ has a sequence as shown in SEQID NO: 2 or a sequence having at least 80, 85, 90, 91, 92, 93, 94, 95, 96, 97, 98 or 99% of identity with SEQ ID NO: 2 or having 1 to 10 modifications selected from the group consisting of addition, deletion, substitution and combinations thereof with respect to the sequence of SEQ ID NO: 2.

Preferably, the molecule comprising a single anti PD-1 or anti-CD28 antigen binding domain and a single SIRP molecule further comprises i) a peptide linker, preferably selected from the group consisting of Gly4SerGly3Ser, (Gly4Ser)4, (Gly4Ser)3, and (Gly3Ser2)3, even more preferably a linker having a sequence selected from the group consisting of SEQ ID NO: 67, SEQ ID NO: 68, SEQ ID NO: 69 and SEQ ID NO: 70 and/or ii) a hinge region.

In addition, such molecule comprises a Fc domain. Preferably, the Fc domain derives from a human IgG1 or IgG4. Even more preferably, the first Fc chain is a hole or H chain and comprises the substitutions T366S/L368A/Y407V/Y349C and optionally N297A and the second Fc chain is a knob or K chain and comprises the substitutions T366W/S354C and optionally N297A. Preferably, the Fc chain is a hole or H chain and comprises the substitutions T366S/L368A/Y407V/Y349C and N297A and the second Fc chain is a knob or K chain and comprises the substitutions T366W/S354C and N297AFor instance, the molecule comprises a second Fc chain comprising or consisting in SEQ ID NO: 14 and a first Fc chain comprising or consisting in SEQ ID NO: 13.

In a very particular aspect, the bifunctional molecule may comprise a first monomer of SEQ ID NO: 14 and a second monomer comprising a Fc chain SEQ ID NO: 13, to which is linked at the N-terminal end, optionally by a linker, to an antigen binding domain (for instance of SEQ ID NO: 12). More preferably, the bifunctional molecule may comprise a first monomer of SEQ ID NO: 14 and a second monomer comprising a Fc chain SEQ ID NO: 13, to which is linked at the N-terminal end, optionally by a linker, to an antigen binding domain (for instance of SEQ ID NO: 12), and at the C-terminal end, optionally by a linker, to any immune-stimulating moiety as disclosed herein, preferably selected from the group consisting of SIRPγ, SIRPα or SIRPβ or any fragments and variants thereof.

Optionally, the immune-stimulating moiety can be selected from the group consisting of the extracellular part of SIRPα (SEQ ID NO: 1) and the extracellular part of SIRPγ (SEQ ID NO: 2), or a variant thereof having at least 80, 85, 90, 91, 92, 93, 94, 95, 96, 97, 98 or 99% of identity therewith or having 1 to 10 modifications selected from the group consisting of addition, deletion, substitution and combinations thereof with respect to the wildtype protein or its extracellular part.

Optionally, when the immune-stimulating moiety is the extracellular part of SIRPγ, the bifunctional molecule may comprise a first monomer of SEQ ID NO: 14, a second monomer of SEQ ID NO: 3, and a third monomer of SEQ ID NO: 37, 38 or 11, preferably SEQ ID NO: 38 or 11.

Optionally, when the immune-stimulating moiety is the extracellular part of SIRPα, the bifunctional molecule may comprise a first monomer of SEQ ID NO: 14, a second monomer of SEQ ID NO: 6, and a third monomer of SEQ ID NO: 37, 38 or 11, preferably SEQ ID NO: 38 or 11.

Optionally, when the immune-stimulating moiety is the extracellular part of SIRPγ, the bifunctional molecule may comprise a first monomer of SEQ ID NO: 14, a second monomer of SEQ ID NO: 9, and a third monomer of SEQ ID NO: 37, 38 or 11, preferably SEQ ID NO: 38 or 11, linked at its terminal end, optionally by a linker, to the extracellular part of SIRPγ of SEQ ID NO: 2 or a variant thereof. More particularly, the bifunctional molecule may comprise a first monomer of SEQ ID NO: 14, a second monomer of SEQ ID NO: 9, and a third monomer of SEQ ID NO: 7. Alternatively, the bifunctional molecule may comprise a first monomer of SEQ ID NO: 13, a second monomer of SEQ ID NO: 10, and a third monomer of SEQ ID NO: 7.

Optionally, when the immune-stimulating moiety is the extracellular part of SIRPα, the bifunctional molecule may comprise a first monomer of SEQ ID NO: 14, a second monomer of SEQ ID NO: 9, and a third monomer of SEQ ID NO: 37, 38 or 11, preferably SEQ ID NO: 38 or 11, linked at its terminal end, optionally by a linker, to the extracellular part of SIRPα of SEQ ID NO: 1 or a variant thereof. More particularly, the bifunctional molecule may comprise a first monomer of SEQ ID NO: 14, a second monomer of SEQ ID NO: 9, and a third monomer of SEQ ID NO: 8. Alternatively, the bifunctional molecule may comprise a first monomer of SEQ ID NO: 13, a second monomer of SEQ ID NO: 10, and a third monomer of SEQ ID NO: 8.

In another very particular aspect, the bifunctional molecule may comprise a first monomer of SEQ ID NO: 13 and a second monomer comprising a Fc chain SEQ ID NO: 14, to which is linked at the N-terminal end, optionally by a linker, to an antigen binding domain (for instance of SEQ ID NO: 12). More preferably, the bifunctional molecule may comprise a first monomer of SEQ ID NO: 13 and a second monomer comprising a Fc chain of SEQ ID NO: 14, to which is linked at the N-terminal end, optionally by a linker, to an antigen binding domain (for instance of SEQ ID NO: 12), and at the C-terminal end, optionally by a linker, to any immune-stimulating moiety as disclosed herein.

Optionally, the immune-stimulating moiety can be selected from the group consisting of the extracellular part of SIRPα (SEQ ID NO: 1) and the extracellular part of SIRPγ (SEQ ID NO: 2), or a variant thereof having at least 80, 85, 90, 91, 92, 93, 94, 95, 96, 97, 98 or 99% of identity therewith or having 1 to 10 modifications selected from the group consisting of addition, deletion, substitution and combinations thereof with respect to the wildtype protein or its extracellular part.

Accordingly, the bifunctional molecule may comprise a first monomer of SEQ ID NO: 13, a second monomer of SEQ ID NO: 15, and a third monomer of SEQ ID NO: 37, 38 or 11, preferably SEQ ID NO: 38 or 11. Particularly, the bifunctional molecule may comprise or consist in a heavy chain, particularly a knob heavy chain, preferably of SEQ ID NO: 15, a complementary Fc chain, preferably a hole Fc chain, preferably of SEQ ID NO: 13 and a light chain, preferably of SEQ ID NO: 37, 38 or 11.

Alternatively, the bifunctional molecule may comprise a first monomer of SEQ ID NO: 13, a second monomer of SEQ ID NO: 75, and a third monomer of SEQ ID NO: 37, 38 or 11, preferably SEQ ID NO: 38 or 11. Particularly, the bifunctional molecule may comprise or consist in a heavy chain, particularly a knob heavy chain, preferably of SEQ ID NO: 75, a complementary Fc chain, preferably a hole Fc chain, preferably of SEQ ID NO: 13 and a light chain, preferably of SEQ ID NO: 37, 38 or 11.

In a particular embodiment, the bifunctional molecule may comprise a first monomer of SEQ ID NO: 84, a second monomer of SEQ ID NO: 85, and a third monomer of SEQ ID NO: 86 or 87. Particularly, the bifunctional molecule may comprise or consist in a heavy chain, particularly a hole heavy chain, preferably of SEQ ID NO: 85, a complementary Fc chain, preferably a knob Fc chain, preferably of SEQ ID NO:84 and a light chain linked to a extracellular part of SIRP, preferably of SEQ ID NO: 86 or 87.

### Preparation of Bifunctional Molecule—Nucleic Acid Molecules Encoding the Bifunctional Molecules of the Present Invention, Recombinant Expression Vectors and Host Cells Comprising such

To produce a bifunctional molecule according to the invention, in particular by mammalian cells, nucleic acid sequences or group of nucleic acid sequences coding for the bifunctional molecule are subcloned into one or more expression vectors. Such vectors are generally used to transfect mammalian cells. General techniques for producing molecules comprising antibody sequences are described in Coligan et al. (eds.), Current protocols in immunology, at pp. 10.19.1-10.19.11 (Wiley Interscience 1992), the contents of which are hereby incorporated by reference and in “Antibody engineering: a practical guide” from W. H. Freeman and Company (1992), in which commentary relevant to production of molecules is dispersed throughout the respective texts.

Generally, such method comprises the following steps of:
(1) transfecting or transforming appropriate host cells with the polynucleotide(s) encoding the recombinant bifunctional molecule of the invention or the vector containing the polynucleotide(s);
(2) culturing the host cells in an appropriate medium; and
(3) optionally isolating or purifying the bifunctional molecule from the medium or host cells.

The invention further relates to a nucleic acid encoding a bifunctional molecule as disclosed above, a vector, preferably an expression vector, comprising the nucleic acid of the invention, a genetically engineered host cell transformed with the vector of the invention or directly with the sequence encoding the recombinant bifunctional molecule, and a method for producing the bifunctional molecule of the invention by recombinant techniques.

The nucleic acid, the vector and the host cells are more particularly described hereafter.

### Nucleic Acid Sequence

The invention also relates to a nucleic acid molecule encoding the bifunctional molecule as defined above or to a group of nucleic acid molecules encoding the bifunctional molecule as defined above. Nucleic acid encoding the bifunctional molecule disclosed herein can be amplified by any techniques known in the art, such as PCR. Such nucleic acid may be readily isolated and sequenced using conventional procedures.

Particularly, the nucleic acid molecules encoding the bifunctional molecule as defined herein comprises:
a first nucleic acid molecule encoding the first monomer comprising an antigen-binding domain covalently linked to a first Fc chain optionally via a peptide linker, said first Fc chain being covalently linked to the immune-stimulating moiety, optionally via a peptide linker, and
a second nucleic acid molecule encoding the second monomer comprising a complementary second Fc chain,
optionally a third nucleic acid molecule encoding the light chain of the antigen-binding domain.

In an alternative aspect, the nucleic acid molecules encoding the bifunctional molecule as defined herein comprises:
a first nucleic acid molecule encoding the first monomer comprising an antigen-binding domain covalently linked to a first Fc chain optionally via a peptide linker,
a second nucleic acid molecule encoding the second monomer comprising a complementary second Fc chain, and
a third nucleic acid molecule encoding the light chain of the antigen-binding domain, said light chain being covalently linked to the immune-stimulating moiety, optionally via a peptide linker.

In one embodiment, the nucleic acid molecule is an isolated, particularly non-natural, nucleic acid molecule.

### Vectors

In another aspect, the invention relates to a vector comprising the nucleic acid molecule or the group of nucleic acid molecules as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The nucleic acid molecule encoding the bifunctional molecule can be cloned into a vector by those skilled in the art, and then transformed into host cells. These methods include in vitro recombinant DNA techniques, DNA synthesis techniques, in vivo recombinant techniques, etc. The methods known to the artisans in the art can be used to construct an expression vector containing the nucleic acid sequence of the bifunctional molecule described herein and appropriate regulatory components for transcription/translation.

Accordingly, the present invention also provides a recombinant vector, which comprises a nucleic acid molecule encoding the bifunctional molecule according to the present invention. In one preferred aspect, the expression vector further comprises a promoter and a nucleic acid sequence encoding a secretion signal peptide, and optionally at least one drug-resistance gene for screening. The expression vector may further comprise a ribosome -binding site for initiating the translation, transcription terminator and the like.

Suitable expression vectors typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

An expression vector can be introduced into host cells using a variety of techniques including calcium phosphate transfection, liposome-mediated transfection, electroporation, and the like. Preferably, transfected cells are selected and propagated wherein the expression vector is stably integrated in the host cell genome to produce stable transformants.

### Host Cells

In another aspect, the invention relates to a host cell comprising a vector or a nucleic acid molecule or group of nucleic acid molecules as defined above, for example for bifunctional molecule production purposes.

As used herein, the term “host cell” is intended to include any individual cell or cell culture that can be or has been recipient of vectors, exogenous nucleic acid molecules, and polynucleotides encoding the bifunctional molecule according to the present invention. The term “host cell” is also intended to include progeny or potential progeny of a single cell. Suitable host cells include prokaryotic or eukaryotic cells, and also include but are not limited to bacteria, yeast cells, fungi cells, plant cells, and animal cells such as insect cells and mammalian cells, e.g., murine, rat, rabbit, macaque or human.

Suitable hosts cells are especially eukaryotic hosts cells which provide suitable post-translational modifications such as glycosylation. Preferably, such suitable eukaryotic host cell may be fungi such as

Pichia pastoris, Saccharomyces cerevisiae, Schizosaccharomyces pombe; insect cell such as Mythimna separate; plant cell such as tobacco, and mammalian cells such as BHK cells, 293 cells, CHO cells, NSO cells and COS cells.

Preferably, the host cell of the present invention is selected from the group consisting of CHO cell, COS cell, NSO cell, and HEK cell.

Then host cells stably or transiently express the bifunctional molecule according to the present invention. Such expression methods are known by the man skilled in the art.

A method of production of the bifunctional molecule is also provided herein. The method comprises culturing a host cell comprising a nucleic acid encoding the bifunctional molecule as provided above, under conditions suitable for its expression, and optionally recovering the bifunctional molecule from the host cell (or host cell culture medium). Particularly, for recombinant production of a bifunctional molecule, nucleic acid encoding a bifunctional molecule, e.g., as described above, is isolated and inserted into one or more vectors for further cloning and/or expression in a host cell. The bifunctional molecules are then isolated and/or purified by any methods known in the art. These methods include, but are not limited to, conventional renaturation treatment, treatment by protein precipitant (such as salt precipitation), centrifugation, cell lysis by osmosis, sonication, supercentrifugation, molecular sieve chromatography or gel chromatography, adsorption chromatography, ion exchange chromatography, HPLC, any other liquid chromatography, and the combination thereof. As described, for example, by Coligan, bifunctional molecule isolation techniques may particularly include affinity chromatography with Protein-A Sepharose, size-exclusion chromatography and ion exchange chromatography. Protein A preferably is used to isolate the bifunctional molecules of the invention.

### Pharmaceutical Composition and Method of Administration Thereof

The present invention also relates to a pharmaceutical composition comprising a bifunctional molecule described herein, the nucleic acid molecule, the group of nucleic acid molecules, the vector and/or the host cells as described hereabove, preferably as the active ingredient or compound. The formulations can be sterilized and, if desired, mixed with auxiliary agents such as pharmaceutically acceptable carriers, excipients, salts, anti-oxidant and/or stabilizers which do not deleteriously interact with the bifunctional molecule of the invention, nucleic acid, vector and/or host cell of the invention and does not impart any undesired toxicological effects. Optionally, the pharmaceutical composition may further comprise an additional therapeutic agent.

Particularly, the pharmaceutical composition according to the invention can be formulated for any conventional route of administration including a topical, enteral, oral, parenteral, intranasal, intravenous, intramuscular, subcutaneous or intraocular administration and the like. To facilitate administration, the bifunctional molecule as described herein can be made into a pharmaceutical composition for in vivo administration. The means of making such a composition have been described in the art (see, for instance, Remington: The Science and Practice of Pharmacy, Lippincott Williams & Wilkins, 21st edition (2005).

The pharmaceutical composition may be prepared by mixing a bifunctional molecule having the desired degree of purity with optional pharmaceutically acceptable carriers, excipients, anti-oxidant, and/or stabilizers in the form of lyophilized formulations or aqueous solutions. Such suitable carriers, excipients, anti-oxidant, and/or stabilizers are well known in the art and have been for example described in Remington's Pharmaceutical Sciences 16th edition, Osol, A. Ed. (1980).

To facilitate delivery, any of the bifunctional molecule or its encoding nucleic acids can be conjugated with a chaperon agent. The chaperon agent can be a naturally occurring substance, such as a protein (e.g., human serum albumin, low-density lipoprotein, or globulin), carbohydrate (e.g., a dextran, pullulan, chitin, chitosan, inulin, cyclodextrin or hyaluronic acid), or lipid. It can also be a recombinant or synthetic molecule, such as a synthetic polymer, e.g., a synthetic polypeptide.

Pharmaceutical compositions according to the invention may be formulated to release the active ingredients (e.g. the bifunctional molecule of the invention) substantially immediately upon administration or at any predetermined time or time period after administration. The pharmaceutical composition in some aspects can employ time-released, delayed release, and sustained release delivery systems such that the delivery of the composition occurs prior to, and with sufficient time to cause, sensitization of the site to be treated. Means known in the art can be used to prevent or minimize release and absorption of the composition until it reaches the target tissue or organ, or to ensure timed-release of the composition. Such systems can avoid repeated administrations of the composition, thereby increasing convenience to the subject and the physician.

It will be understood by one skilled in the art that the formulations of the invention may be isotonic with human blood that is the formulations of the invention have essentially the same osmotic pressure as human blood. Such isotonic formulations generally have an osmotic pressure from about 250 mOSm to about 350 mOSm. Isotonicity can be measured by, for example, a vapor pressure or ice-freezing type osmometer.

Pharmaceutical composition typically must be sterile and stable under the conditions of manufacture and storage. Prevention of presence of microorganisms may be ensured both by sterilization procedures (for example by microfiltration), and/or by the inclusion of various antibacterial and antifungal agents

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect.

### Subject, Regimen and Administration

The present invention relates to a bifunctional molecule as disclosed herein, a nucleic acid or a vector encoding such, a host cell or a pharmaceutical composition for use as a medicament or for use in the treatment of a disease or for administration in a subject or for use as a medicament. It also relates to a method for treating a disease or a disorder in a subject comprising administering a therapeutically effective amount of a pharmaceutical composition or a bifunctional molecule to a subject.

The subject to treat may be a human, particularly a human at the prenatal stage, a new-born, a child, an infant, an adolescent or an adult, in particular an adult of at least 30 years old, 40 years old, preferably an adult of at least 50 years old, still more preferably an adult of at least 60 years old, even more preferably an adult of at least 70 years old.

In a particular aspect, the subject can be immunosuppressed or immunocompromised.

Conventional methods, known to those of ordinary skill in the art of medicine, can be used to administer the bifunctional molecule or the pharmaceutical composition disclosed herein to a subject, depending upon the type of diseases to be treated or the site of the disease e.g., administered orally, parenterally, enterally, by inhalation spray, topically, rectally, nasally, buccally, vaginally or via an implanted reservoir. Preferably, the bifunctional molecule or the pharmaceutical composition is administered via subcutaneous, intra-cutaneous, intravenous, intramuscular, intra-articular, intra-arterial, intra-synovial, intra-tumoral, intra-sternal, intra-thecal, intra-lesion, and intracranial injection or infusion techniques.

The form of the pharmaceutical compositions, the route of administration and the dose of administration of the pharmaceutical composition or the bifunctional molecule according to the invention can be adjusted by the man skilled in the art according to the type and severity of the infection, and to the patient, in particular its age, weight, size, sex, and/or general physical condition. The compositions of the present invention may be administered in a number of ways depending upon whether local or systemic treatment is desired.

### Use in the Treatment of a Disease

The bifunctional molecules, nucleic acids, vectors, host cells, compositions and methods of the present invention have numerous in vitro and in vivo utilities and applications. Particularly, any of bifunctional molecules, nucleic acid molecules, group of nucleic acid molecules, vectors, host cells or pharmaceutical composition provided herein may be used in therapeutic methods and/or for therapeutic purposes.

The present invention also relates to a bifunctional molecule, a nucleic acid or a vector encoding such, or a pharmaceutical composition comprising such for use in the treatment of a disorder and/or disease in a subject and/or for use as a medicament or vaccine. It also relates to the use of a bifunctional molecule as described herein; a nucleic acid or a vector encoding such, or a pharmaceutical composition comprising such for treating a disease and/or disorder in a subject. Finally, it relates to a method for treating a disease or a disorder in a subject comprising administering a therapeutically effective amount of a pharmaceutical composition or a bifunctional molecule to the subject, or a nucleic acid or a vector encoding such.

In one aspect, the invention relates to a method of treatment of a disease and/or disorder selected from the group consisting of a cancer, an infectious disease and a chronic viral infection in a subject in need thereof comprising administering to said subject an effective amount of a bifunctional molecule or pharmaceutical composition as defined above. Examples of such diseases are more particularly described hereafter.

In one aspect, the treatment method comprises: (a) identifying a patient in need of treatment; and (b) administering to the patient a therapeutically effective amount of a bifunctional molecule, nucleic acid, vector or pharmaceutical composition as described herein.

A subject in need of a treatment may be a human having, at risk for, or suspected of having a disease. Such a patient can be identified by routine medical examination.

In another aspect, the bifunctional molecules disclosed herein can be administered to a subject, e.g., in vivo, to enhance immunity, preferably in order to treat a disorder and/or disease. Accordingly, in one aspect, the invention provides a method of modifying an immune response in a subject comprising administering to the subject a bifunctional molecule, nucleic acid, vector or pharmaceutical composition of the invention such that the immune response in the subject is modified. Preferably, the immune response is enhanced, increased, stimulated or up-regulated. The bifunctional molecule or pharmaceutical composition can be used to enhance immune responses such as T cell activation in a subject in need of a treatment. In a particular embodiment, the bifunctional molecule or pharmaceutical composition can be used to reduce T cells exhaustion or to reactivate exhausted T cells.

The invention particularly provides a method of enhancing an immune response in a subject, comprising administering to the subject a therapeutic effective amount of any of the bifunctional molecule, nucleic acid, vector or pharmaceutical composition comprising such described herein, such that an immune response in the subject is enhanced. In a particular embodiment, the bifunctional molecule or pharmaceutical composition can be used to reduce T cells exhaustion or to reactivate exhausted T cells.

Bifunctional molecules including SIRP according to the invention target CD47+ immune cells, particularly CD47+ T cells. Such cells may be found in the following areas of particular interest: resident lymphoid cells in the lymph nodes (mainly within paracortex, with occasional cells in follicles), in tonsil (inter-follicular areas), spleen (mainly within the Peri-Arteriolar Lymphoid Sheaths (PALS) of the white pulp and some scattered cells in the red pulp), thymus (primarily in medulla; also in cortex), bone marrow (scattered distribution), in the GALT (Gut Associated-Lymphoid-Tissue, primarily in inter-follicular areas and lamina propria) throughout the digestive tract (stomach, duodenum, jejunum, ileum, cecum colon, rectum), in the MALT (Mucosa-Associated-Lymphoid-Tissue) of the gall bladder. Therefore, the bifunctional molecules of the invention are of particular interest for treating diseases located or involving these areas, in particular cancers.

### Cancer

In another aspect, the invention provides the use of a bifunctional molecule or pharmaceutical composition as disclosed herein in the manufacture of a medicament for treating a cancer, for instance for inhibiting growth of tumor cells in a subject.

The term “cancer” as used herein is defined as disease characterized by the rapid and uncontrolled growth of aberrant cells. Cancer cells can spread locally or through the bloodstream and lymphatic system to other parts of the body.

Accordingly, in one aspect, the invention provides a method of treating a cancer, for instance for inhibiting growth of tumor cells, in a subject, comprising administering to the subject a therapeutically effective amount of bifunctional molecule or pharmaceutical composition according to the invention. Particularly, the present invention relates to the treatment of a subject using a bifunctional molecule such that growth of cancerous cells is inhibited.

In an aspect of the disclosure, the cancer to be treated is associated with exhausted T cells.

Any suitable cancer may be treated with the provided herein can be hematopoietic cancer or solid cancer. Such cancers include carcinoma, cervical cancer, colorectal cancer, esophageal cancer, gastric cancer, gastrointestinal cancer, head and neck cancer, kidney cancer, liver cancer, lung cancer, lymphoma, glioma, mesothelioma, melanoma, stomach cancer, urethral cancer environmentally induced cancers and any combinations of said cancers. Additionally, the invention includes refractory or recurrent malignancies. Preferably, the cancer to be treated or prevented is selected from the group consisting of metastatic or not metastatic, Melanoma , malignant mesothelioma, Non-Small Cell Lung Cancer, Renal Cell Carcinoma, Hodgkin's Lymphoma, Head and Neck Cancer, Urothelial Carcinoma, Colorectal Cancer, Hepatocellular Carcinoma, Small Cell Lung Cancer Metastatic Merkel Cell Carcinoma, Gastric or Gastroesophageal cancers and Cervical Cancer.

In a particular aspect, the cancer is a hematologic malignancy or a solid tumor. Such a cancer can be selected from the group consisting of hematolymphoid neoplasms, angioimmunoblastic T cell lymphoma, myelodysplasic syndrome, acute myeloid leukemia.

In a particular aspect, the cancer is a cancer induced by virus or associated with immunodeficiency. Such a cancer can be selected from the group consisting of Kaposi sarcoma (e.g., associated with Kaposi sarcoma herpes virus); cervical, anal, penile and vulvar squamous cell cancer and oropharyndeal cancers (e.g., associated with human papilloma virus); B cell non-Hodgkin lymphomas (NHL) including diffuse large B-cell lymphoma, Burkitt lymphoma, plasmablastic lymphoma, primary central nervous system lymphoma, HHV-8 primary effusion lymphoma, classic Hodgkin lymphoma, and lymphoproliferative disorders (e.g., associated with Epstein-Barr virus (EBV) and/or Kaposi sarcoma herpes virus); hepatocellular carcinoma (e.g., associated with hepatitis B and/or C viruses); Merkel cell carcinoma (e.g., associated with Merkel cell polyoma virus (MPV)); and cancer associated with human immunodeficiency virus infection (HIV) infection.

Preferred cancers for treatment include cancers typically responsive to immunotherapy. Alternatively, preferred cancers for treatment are cancers non-responsive to immunotherapy.

### Infectious Disease

The bifunctional molecule, nucleic acid, group of nucleic acid, vector, host cells or pharmaceutical compositions of the invention can be used to treat patients that have been exposed to particular toxins or pathogens. Accordingly, an aspect of the invention provides a method of treating an infectious disease in a subject comprising administering to the subject a bifunctional molecule according to the present invention, or a pharmaceutical composition comprising such, preferably such that the subject is treated for the infectious disease.

Any suitable infection may be treated with a bifunctional molecule, nucleic acid, group of nucleic acid, vector, host cells or pharmaceutical composition as provided herein.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the invention include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

### Combined Therapy

The bifunctional molecule according to the invention can be combined with some other potential strategies for overcoming immune evasion mechanisms with agents in clinical development or already on the market (see table 1 from Antonia et al. Immuno-oncology combinations: a review of clinical experience and future prospects. Clin. Cancer Res. Off. J. Am. Assoc. Cancer Res. 20, 6258-6268, 2014). Such combination with the bifunctional molecule according to the invention may be useful notably for:
1—Reversing the inhibition of adaptive immunity (blocking T-cell checkpoint pathways);
2—Switching on adaptive immunity (promoting T-cell costimulatory receptor signaling using agonist molecules, in particular antibodies),
3—Improving the function of innate immune cells;
4—Activating the immune system (potentiating immune-cell effector function), for example through vaccine-based strategies.

Accordingly, also provided herein are combined therapies with any of the bifunctional molecule or pharmaceutical composition comprising such, as described herein and a suitable second agent, for the treatment of a disease or disorder. In an aspect, the bifunctional molecule and the second agent can be present in a unique pharmaceutical composition as described above. Alternatively, the terms “combination therapy” or “combined therapy”, as used herein, embrace administration of these two agents (e.g., a bifunctional molecule as described herein and an additional or second suitable therapeutic agent) in a sequential manner, that is, wherein each therapeutic agent is administered at a different time, as well as administration of these therapeutic agents, or at least two of the agents, in a substantially simultaneous manner. Sequential or substantially simultaneous administration of each agent can be affected by any appropriate route. The agents can be administered by the same route or by different routes. For example, a first agent (e.g., a bifunctional molecule) can be administered orally, and an additional therapeutic agent (e.g., an anti-cancer agent, an anti-infection agent; or an immune modulator) can be administered intravenously. Alternatively, an agent of the combination selected may be administered by intravenous injection while the other agents of the combination may be administered orally.

In an aspect, the additional therapeutic agent can be selected in the non-exhaustive list comprising alkylating agents, angiogenesis inhibitors, antibodies, antimetabolites, antimitotics, antiproliferatives, antivirals, aurora kinase inhibitors, apoptosis promoters (for example, Bcl-2 family inhibitors), activators of death receptor pathway, Bcr-Abl kinase inhibitors, BiTE (Bi-Specific T cell Engager) antibodies, antibody drug conjugates, biologic response modifiers, Bruton's tyrosine kinase (BTK) inhibitors, cyclin-dependent kinase inhibitors, cell cycle inhibitors, cyclooxygenase-2 inhibitors, DVDs, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNAs, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids/deltoids plant alkaloids, small inhibitory ribonucleic acids (siRNAs), topoisomerase inhibitors, ubiquitin ligase inhibitors, hypomethylating agents, checkpoints inhibitors, peptide vaccine and the like, epitopes or neoepitopes from tumor antigens, as well as combinations of one or more of these agents.

For instance, the additional therapeutic agent can be selected in the group consisting of chemotherapy, radiotherapy, targeted therapy, antiangiogenic agents, hypomethylating agents, cancer vaccines, epitopes or neoepitopes from tumor antigens, myeloid checkpoints inhibitors, other immunotherapies, and HDAC inhibitors.

In an embodiment, the invention relates to a combined therapy as defined above, wherein the second therapeutic agent is particularly selected from the group consisting of therapeutic vaccines, immune checkpoint blockers or activators, in particular of adaptive immune cells (T and B lymphocytes) and antibody-drug conjugates. Preferably, suitable agents for co-use with any of the anti-hPD-1 antibodies or fragment thereof or with the pharmaceutical composition according to the invention include an antibody binding to a co-stimulatory receptor (e.g., OX40, CD40, ICOS, CD27, HVEM or GITR), an agent that induces immunogenic cell death (e.g., a chemotherapeutic agent, a radio-therapeutic agent, an anti-angiogenic agent, or an agent for targeted therapies), an agent that inhibits a checkpoint molecule (e.g., CTLA4, LAG3, TIM3, B7H3, B7H4, BTLA, or TIGIT), a cancer vaccine, an agent that modifies an immunosuppressive enzyme (e.g., IDO1 or iNOS), an agent that targets Treg cells, an agent for adoptive cell therapy, or an agent that modulates myeloid cells.

In an embodiment, the invention relates to a combined therapy as defined above, wherein the second therapeutic agent is an immune checkpoint blocker or activator of adaptive immune cells (T and B lymphocytes) selected from the group consisting of anti-CTLA4, anti-CD2, anti-CD28, anti-CD40, anti-HVEM, anti-BTLA, anti-CD160, anti-TIGIT, anti-TIM-1/3, anti-LAG-3, anti-2B4, and anti-OX40, anti-CD40 agonist, CD40-L, TLR agonists, anti-ICOS, ICOS-L and B-cell receptor agonists.

The present invention also relates to a method for treating a disease in a subject comprising administering to said subject a therapeutically effective amount of the bifunctional molecule or the pharmaceutical composition described herein and a therapeutically effective amount of an additional or second therapeutic agent.

Specific examples of additional or second therapeutic agents are provided in WO 2018/053106, pages 36-43.

In a preferred embodiment, the second therapeutic agent is selected from the group consisting of chemotherapeutic agents, radiotherapy agents, immunotherapeutic agents, cell therapy agents (such as CAR-T cells), antibiotics and probiotics.

Combination therapy could also rely on the combination of the administration of bifunctional molecule with surgery.

### EXAMPLES

### Example 1: Description of the Constructions used in the Example 2 to 5.

The inventors designed and compared the biological activity of multiple structures of bifunctional molecules comprising one or two anti PD-1 binding domains and one SIRPg protein fused to the C terminal domain of the anti PD-1 antibody as described in FIG. 1.

### Example 2. Bifunctional Antibodies Constructed with One Anti PD-1 Valency Fused to One SIRP Protein Demonstrated Higher Productivity by Mammalian Cells Compared To Bifunctional Antibody Constructed with Two Anti PD-1 Valency Fused to One SIRP Protein

The productivity of the bifunctional antibodies by mammalian cells was assessed and compared. Full Heavy chain with a Fc fused to type I protein (SIRPg) and the Fc chain were transiently co-transfected with the light chains into CHO suspension cells. Quantity of antibody obtained after production and purification was quantified using a sandwich ELISA (immobilized donkey anti human Fc antibody for detection and revelation with a mouse anti human kappa+a peroxidase conjugated goat anti mouse antibody). Concentration was determined with human lvlgG standard. Productivity was calculated as the quantity of purified antibody per liter of collected culture supernatant.

Results: Bifunctional antibodies, anti PD-1*2/SIRPg*1 and antibody PD-1*1/SIRPg*1 were produced in CHO mammalian and the results presented in FIG. 2. In a surprising manner, the anti PD-1*1/SIRPg*1 construction has a better productive yield (mg/L) than the anti PD-1*2/SIRPg*1. This result indicates that the anti PD-1*1/SIRPg*1 presents a very good manufacturability which is very important for the next steps of the clinical development and therapeutic applications, especially for heterodimeric bifunctional antibodies that have low yield of productivity (Wang et al, 2019, Antibodies, 8, 43; Wei et al, 2017, Oncotarget, 8, 51037-51049).

In fact, the productivity yield of the anti PD-1*1/SIRPg*1 is in a similar range than an anti PD-1 alone (anti PD-1*1 or anti PD-1*2), the productivity of which is equal to 45 mg/L (n=5) in similar conditions of production. This facilitates the development as therapeutic agent for large scale productivity.

### Example 3: Competitive Assays to Measure the Activity of the Anti-PD1*1/SIRP*1 Bifunctional Antibody and its Capacity to Antagonize PD-1/PD-L1 Interaction

The binding of anti PD-1*1/SIRPg*1 to bind to human PD-1 receptor and to antagonize PD-L1/PD-1 interaction was measured by ELISA assays. Recombinant hPD1 (Sino Biologicals, Beijing, China; reference 10377-H08H) was immobilized on plastic at 0.5 g/ml in carbonate buffer (pH 9.2) and purified antibody was added to measure binding. After incubation and washing, peroxidase-labeled donkey anti-human lgG (Jackson Immunoresearch; USA; reference 709-035-149) was added and revealed by conventional methods.

To measure antagonist activity of the bifunctional antibody, a competitive ELISA assay was performed by PD-1:PD-L1 Inhibitor Screening ELISA Assay Pair (AcroBiosystems; USA; reference EP-101). In this assay, recombinant hPDL1 was immobilized on plastic at 2 μg/ml in PBS pH 7.4 buffer. Purified antibodies (at different concentrations) were mixed with 0.66 μg/ml final (fix concentration) of biotinylated Human PD1 (AcroBiosystems; USA; reference EP-101) to measure competitive binding for 2h at 37° C. After incubation and washing, peroxidase-labeled streptavidin (Vector laboratoring; USA; reference SA-5004) was added to detect the c binding and revealed by conventional methods.

Results: FIG. 3 and table 1 show that the bifunctional anti PD-1*1/SIRPg*1 molecule demonstrates an efficient PD-1 binding, demonstrating that even with one anti PD-1 valency, conserve a good binding to PD-1 antigen. Moreover, the antagonist activity is also preserved as shown in Table 2.

TABLE 1
EC50 (nM) of the PD-1 Binding from the FIG. 3.

| PD-1 binding | EC50 (nM) |
|---|---|
| anti PD-1*1/SIRPg*1 | 0.33 |

TABLE 2
EC50 (nM) Competition PD-1/PD-L1 ELISA assay.

| PD-1/PD-L1 antagonist | IC50 (nM) |
|---|---|
| anti PD-1*1/SIRPg*1 | 6.9 |

### Example 4: Anti PD-1*1 SIRPg*1 Demonstrated Better Pharmacokinetics Profile in vivo Compared to the Anti PD-1*1 SIRPg*2, or the Anti PD-1*2 SIRPg*2 Molecules

Pharmacokinetics and Pharmacodynamics of the product were assessed in mice following a single injection. C57bl6JRj mice (female 6-9 weeks) were intravenously or intraperitoneally injected with a single dose (34 nmol/kg) of anti PD-1 or bifunctional antibodies. Plasma drug concentration was determined by ELISA using an immobilized anti-human light chain antibody (clone NaM76-5F3), then serum-containing antibodies were added. Detection was performed with a peroxidase-labeled donkey anti-human lgG (Jackson Immunoresearch; USA; reference 709-035-149) was added and revealed by conventional methods. Area under the Curve corresponding to the drug exposure was calculated for each construction.

Results: FIG. 4 shows the pharmacokinetics profile of the anti PD-1/SIRPg constructed with 1 or two SIRPg protein and 1 or 2 anti PD-1 valencies after a single intravenous injection. The anti PD-1*1/SIRPg*1 demonstrated the best pharmacokinetics profile in comparison to the anti PD-1*2/SIRPg*2 and the anti PD-1*2/SIRPg*1. Right graphics of the FIG. 4 shows the Area under the curve of the pharmacokinetics experiments allowing to compare between the different construction the in vivo drug exposure. Similarly, a higher AUC was calculated for the anti PD-1*1/SIRPg*1 construction.

In another experiments, pharmacokinetics of anti PD-1*2 or anti PD-1*1 antibody alone was also assessed to understand whether the anti PD-1 construction alone allows a better pharmacokinetics profile or whether this observation is only applicable to bifunctional molecules. FIG. 5 shows that both anti PD-1*2 and anti PD-1*1 have similar profile after intravenous (FIG. 5A) or intraperitoneal (FIG. 5B) injection, suggesting that, surprisingly, the anti PD-1*1 construction induces a better pharmacokinetics profile only for bifunctional molecules.

The poor pharmacokinetics profile is a well-known challenge for bifunctional antibodies. Bifunctional antibodies are rapidly eliminated and present a short half-life in vivo limiting their use in clinic. The format anti PD-1*1/SIRPg*1 of the present invention allows to improve pharmacokinetics profile in vivo, with longer term exposure compared to others formats Anti PD-1*2/SIRPg*2 or anti PD-1*2/SIRPg*1 antibodies.

### Example 5: The Anti PD-1*1/SIRPg*1 Bifunctional Molecule Promotes Phagocytosis of CD47+ Tumor Cells

Phagocytosis assay was performed by coculturing huH7 hepatocarcinoma human tumor cells with human M1 macrophages that are labeled with 2 different intracellular cell tracker dyes. The percentage of phagocytosis was quantified by flow cytometry following 45 minutes of coincubation with isotype control, anti PD-1*2 or Anti PD-1*1/SIRPg*1 antibody. Phagocytosis of 2 tumor cell lines were tested, HuH7 CD47 negative cells or HuH7 transduced to stably express human CD47.

Results: FIG. 6 shows that anti PD-1*1/SIRPg*1 significantly enhances phagocytosis of CD47 expressing HuH7 tumor cells (right graph) by M1 macrophages to higher extent compared to the anti PD-1-alone. No phagocytosis was measured CD47 negative tumor cells with the anti PD-1*1/SIRPg*1 molecule (FIG. 6 left graph), demonstrating that the effect observed on the tumor cell phagocytosis is mediated by the targeting of CD47 molecule on tumor cells by the anti PD-1/SIRPg bifunctional antibody.

### Example 6: Anti CD28/SIRP Bispecific Antibodies Efficiently Bind to CD28

The binding of the bispecific antibodies was quantified using MSD technology. Human CD28-Fc was coated to the well (R&D #342-CD) at 5 μg/mL (MSD plate), then anti-CD28/SIRPα (▴) or anti CD28/SIRPg (▾) bispecific molecules were added at serial dilutions. As positive control, an anti CD28 antibody with 2 paratopes (A) was tested and an isotype was used as negative control (grey line). Binding of antibodies was revealed using an anti-human sulfo-tag Kappa antibody (dilution 1 to 1000).

Results: FIG. 7 shows that the bifunctional anti CD28*1/SIRP*1 molecule demonstrates an efficient CD28 binding, showing that even a molecule having one valency conserves a good binding to CD28 antigen.

### Example 7: Anti CD28*1/SIRP*1 Bifunctional Molecule Promotes T Cell Activation

T cell activation assay was performed by measuring IL-2 secretion of CD28+ cells. Jurkat CD28+ T cell line (1^{e}5 cells) were plated on CD3 (A) or CD3+ CD47 Fc (B) coated plates. CD3 (OKT3 1 μg/mL coating) and CD47-Fc (Sinobiological #12283 10 μg/mL coating). Bispecific antibodies, SIRPA or SIRPG were incubated at 100 nM. AncCD28.1 (superagonist anti-CD28 antibody) was used as positive control of IL-2 secretion. 48 h after stimulation, IL-2 secretion was quantified by ELISA in culture supernatant.

Results: FIG. 8 shows the capacity of anti CD28*1/SIRP*1 to specifically and significantly promote T cell activation bound through CD3/CD47Fc wells, far more efficiently than SIRP-Fc counterparts (B), while CD28+ cells bound only by CD3 did not secrete IL2 (A), proving the efficacity of anti CD28*1/SIRP*1 to promote T cell activation. Such capacity seems to be mediated through binding of CD47 and trans activation of T cells. In this assay coating of CD47 mimics overexpression of CD47 on tumor cells.

### Material and Methods

### ELISA Binding PD1

For activity ELISA assay, recombinant hPD1 (Sino Biologicals, Beijing, China; reference 10377-H08H) was immobilized on plastic at 0.5 μg/ml in carbonate buffer (pH 9.2) and purified antibody were added to measure binding. After incubation and washing, peroxidase-labeled donkey anti-human IgG (Jackson Immunoresearch; USA; reference 709-035-149) was added and revealed by conventional methods.

### ELISA Antagonist: Competition Between PDL1 and Humanized Anti-PD1

Competitive ELISA assay was performed by PD-1:PD-L1 Inhibitor Screening ELISA Assay Pair (Acro Biosystems; USA; reference EP-101). In this assay, recombinant hPDL1 was immobilized on plastic at 2 μg/ml in PBS pH 7.4 buffer. Purified antibodies(at different concentrations) were mixed with 0.66 μg/ml final (fix concentration) of biotinylated Human PD1 (AcroBiosystems; USA; reference EP-101) to measure competitive binding for 2h at 37° C. After incubation and washing, peroxidase-labeled streptavidin (Vector laboratoring; USA; reference SA-5004) was added to detect Biotin-PD-1Fc binding and revealed by conventional methods.

Pharmacokinetics of the Anti PD-1/SIRPg in vivo

To analyze the pharmacokinetics, a single dose of the molecule was intra-orbitally or intraperitoneally or intravenously (retroorbital) injected into C57bl6JrJ mice (female 6-9 weeks) Drug concentration in the plasma was determined by ELISA using an immobilized anti-human light chain antibody (clone NaM76-5F3) Detection was performed with a peroxidase-labeled donkey anti-human IgG (Jackson Immunoresearch; USA; reference 709-035-149) and revealed by conventional methods.

Phagocytosis of the Tumor Cells with Human Macrophages

Monocytes from human PBMCs were isolated with CD14+ magnetic beads (#130-050-201 Miltenyi) and differentiated into M1 macrophages. M1 macrophages were labelled with cell tracker green (dilution 1 to 2000) and CD47 negative or CD47 transduced HuH7 cells were labeled with a cell tracker red (dilution 1 to 2000). M1 macrophages and tumor cells were cocultivated (ratio 1:2) with an isotype control, the anti PD-1*2 or the anti PD-1*1/SIRPg*1 for 45 minutes in ultralow attachment plate. Cells were then fixed with PFA 1% diluted in PBS for 15 minutes to stop the reaction. Phagocytosis was quantified by flow cytometry (% cell tracker red tumor cells in Cell tracker green+M1 macrophages) and data were normalized to the isotype control group.

### Antibodies and Bifunctional Molecules

The following antibodies and bifunctional molecules have been used in the different experiments disclosed herein.

| | SEQ ID NO | SEQ ID NO | SEQ ID NO |
|---|---|---|---|
| | (if relevant, | (if relevant, | Light |
| Molecule | Chain A) | Chain B) | Chain |
| antiPD-1*2 SIRPG*2 | 4 | — | 11 |
| antiPD-1*2 SIRPG*1 | 10 | 3 | 11 |
| antiPD-1*1 SIRPG*1 | 14 | 3 | 11 |
| antiPD-1*2 SIRPA*2 | 5 | — | 11 |
| antiPD-1*2 SIRPA*1 | 10 | 6 | 11 |
| antiPD-1*1 SIRPA*1 | 14 | 6 | 11 |
| antiPD-1*1 SIRPG*1 | 14 | 9 | 7 |
| (on light chain) | | | |
| antiPD-1*1 SIRPA*1 | 14 | 9 | 8 |
| (on light chain) | | | |
| antiCD28*1 SIRPA*1 | 84 | 85 | 86 |
| antiCD28*1 SIRPG*1 | 84 | 85 | 87 |

1-16. (canceled).

17. A bifunctional molecule comprising a single antigen binding domain that binds to a target specifically expressed on immune cells surface and a single immune-stimulating moiety,
wherein the molecule comprises a first monomer comprising an antigen-binding domain covalently linked via C-terminal end to N-terminal end of a first Fc chain, optionally via a peptide linker, and a second monomer comprising a complementary second Fc chain devoid of antigen-binding domain and of the immune-stimulating moiety;
wherein either i) the immune-stimulating moiety is covalently linked to the C-terminal end of said first Fc chain, optionally via a peptide linker; or ii) the single antigen binding domain comprises a heavy variable chain and a light variable chain and the immune-stimulating moiety is covalently linked to the C-terminal end of the light chain;
wherein the target specifically expressed on immune cells surface is selected from the group consisting of PD-1, CD28, CTLA-4, BTLA, TIGIT, CD160, CD40L, ICOS, CD27, OX40, 4-1BB, GITR, HVEM, Tim-1, LFA-1, TIM3, CD39, CD30, NKG2D, LAG3, B7-1, 2B4, DR3, CD101, CD44, SIRPG, CD28H, CD38, CD3, PDL2, and PDL1; and
wherein the immune-stimulating moiety is selected from the group consisting of SIRPγ, SIRPα, SIRPβ, CD80, CD86, LIGHT, CTLA-4, TIGIT, CD40L, OX40L, APRIL and GITRL or a fragment thereof comprising the extracellular part thereof or a variant thereof having at least 80% of identity with the wildtype protein or the extracellular part thereof.

18. The bifunctional molecule of claim 17, wherein the immune-stimulating moiety is SIRPα, or a fragment thereof comprising the extracellular part thereof or a variant thereof having at least 80% of identity with the wildtype protein or the extracellular fragment thereof.

19. The bifunctional molecule of claim 17, wherein the immune-stimulating moiety is SIRPα, SIRPγ, and or a fragment thereof comprising the extracellular part thereof or a variant thereof having at least 80% of identity with the wildtype protein or the extracellular fragment thereof.

20. The bifunctional molecule of claim 17, wherein the immune-stimulating moiety is linked at the C-terminal end of first Fc chain.

21. The bifunctional molecule of claim 17, wherein the immune-stimulating moiety is linked at the C-terminal end of the light chain.

22. The bifunctional molecule of claim 17, wherein the first Fc chain and the second Fc chain form a heterodimeric Fc domain.

23. The bifunctional molecule of claim 17, wherein the antigen-binding domain is a Fab domain, a Fab′, a single-chain variable fragment (scFV) or a single domain antibody (sdAb).

24. The bifunctional molecule of claim 17, wherein the target specifically expressed on immune cells surface is selected from the group consisting of PD-1, CD28, CTLA-4, BTLA, TIGIT, LAG3 and TIM3.

25. The bifunctional molecule of claim 17, wherein the antigen binding domain binds to PD-1.

26. The bifunctional molecule of claim 17, wherein the antigen binding domain comprises: (i) a heavy chain comprising a CDR1 of SEQ ID NO: 51, a CDR2 of SEQ ID NO: 53 and a CDR3 of SEQ ID NO: 55, 56, 57, 58, 59, 60, 61 or 62; and (ii) a light chain comprising a CDR1 of SEQ ID NO: 64 or 65, a CDR2 of SEQ ID NO: 66 and a CDR3 of SEQ ID NO: 16.

27. The bifunctional molecule of claim 17, wherein the antigen binding domain binds to CD28.

28. The bifunctional molecule of claim 17, wherein the antigen binding domain comprises: (i) a heavy chain comprising a CDR1 of SEQ ID NO: 77, a CDR2 of SEQ ID NO: 78 and a CDR3 of SEQ ID NO: 79; and (ii) a light chain comprising a CDR1 of SEQ ID NO: 81, a CDR2 of SEQ ID NO: 82 and a CDR3 of SEQ ID NO: 83.

29. An isolated nucleic acid sequence or a group of isolated nucleic acid molecules encoding the bifunctional molecule according to claim 17.

30. A host cell comprising the isolated nucleic acid according to claim 29.

31. A pharmaceutical composition comprising the bifunctional molecule according to claim 17 and a pharmaceutically acceptable carrier.

32. A method of treating cancer or an infection disease comprising administering a bifunctional molecule according to claim 17 to a subject having cancer or an infectious disease.
